(19) United States
(12) Patent Application Publication
(10) Pub. No.: US 2012/0149647 A1
(43) Pub. Date: Jun. 14, 2012

(54) Methods for Assessing the Efficacy of Gemcitabine or Ara-C Treatment of Cancer Using Human Antigen R Levels

(76) Inventors: Jonathan R. Brody, Philadelphia, PA (US); Agnieszka K. Witkiewicz, Rydal, PA (US)

(21) Appl. No.: 13/257,449

(22) PCT Filed: Mar. 17, 2010
(86) PCT No.: PCT/US10/27734

§ 371 Date: Feb. 28, 2012

Related U.S. Application Data
(60) Provisional application No. 61/160,937, filed on Mar. 17, 2009.

(51) Int. Cl. A61K 38/17; A61P 35/02; A61K 31/7088; A61P 35/00; G01N 33/567; C12Q 1/68

(52) U.S. Cl. 514/19.6; 435/7.23; 435/6.12; 514/19.3; 514/44 R

(57) ABSTRACT

Disclosed are compositions and methods relating to the treatment of a disease with a nucleoside analog, such as gemcitabine or Ara-C, and a polynucleotide construct encoding for an mRNA binding protein, such as Human antigen R.

### 1. PRIORITY DATA

This application claims priority to U.S. Application Ser. No. 61/160,937, filed Mar. 17, 2009, which is hereby incorporated by reference in its entirety.

### 2. BACKGROUND

The search for treatments for cancers continues to be one of the greatest scientific endeavors. Though many therapies have been developed, there are many types of cancers for which adequate treatments are available for a large number of people or animals. For example, worldwide, 213,000 patients will develop pancreatic ductal adenocarcinoma (PDA) in 2008 and nearly all will die of their disease. Only surgery has modest success with this lethal disease though only 20% of patients are candidates for surgery and of those only 20% will survive 5 years. Emerging targeted drug therapies have welded disappointing results for the treatment of PDA. However, clinical trials did not select for patients predicted to respond to novel or conventional targeted therapies.

Many promising anticancer drugs target specific regulatory proteins and will be effective only in specific subsets of patients. Stratifying patients into likely and unlikely responders to new or existing drugs is a major challenge. Comprehensive co-expression profiles of target proteins and their cofactors across large numbers of cancers will help stratify patients into groups according to predicted responsiveness to existing, new, and forthcoming targeted therapies. Reliable quantitative tissue profiling of proteins is needed to help identify patients who are most likely to benefit from a particular agent. Additionally, targeted therapies for cancer are needed.

### 3. SUMMARY

The present invention, in one embodiment, is directed to a method of assessing the efficacy of gemcitabine treatment of cancer in a subject comprising examining a biological sample from the subject, measuring the expression level and/or activity level of Human Antigen R (HuR) in the sample, and identifying the subject as resistant to or responsive to gemcitabine treatment. In another embodiment, an elevated level of HuR in the cells relative to normal cells or a non-responding subject indicates that the subject is responsive to genicitabine treatment. In another embodiment, the HuR is cytoplasmic HuR. In one embodiment of the invention, an elevated expression level or activity level of HuR is correlated with responsiveness to gemcitabine treatment. In yet another embodiment, a negative expression or activity level of HuR relative to normal cells or cells of a non-responding subject is correlated with resistance to gemcitabine treatment. The biological sample may be a tumor sample from a biopsy or surgical resection. The level of expression and/or activity of HuR may be measured by immunohistochemistry, immunoprecipitation, or real time PCR. In another embodiment, the subject may suffer from pancreatic cancer, small cell lung cancer, colorectal, head and neck cancer, ovarian cancer, melanoma, renal cell carcinoma, non-small cell lung cancer, bladder cancer, ooesophageal cancer, lymphoma, leukemia, or gastric cancer.

Another embodiment of the invention is directed to a method of enhancing the efficacy of gemcitabine treatment of a cancer subject comprising increasing the expression level of HuR in said subject. In this embodiment, the HuR may be cytoplasmic HuR. In one embodiment, the cancer subject is co-administered gemcitabine and a polynucleotide construct encoding for HuR. In another embodiment, the subject is first administered a polynucleotide construct encoding for HuR and then gemcitabine is administered in yet another embodiment, the subject is first administered gemcitabine and then administered a polynucleotide construct encoding for HuR. The subject may have pancreatic cancer, small cell lung cancer, colorectal, head and neck cancer, ovarian cancer, melanoma, renal cell carcinoma, non-small cell lung cancer, bladder cancer, ooesophageal cancer, lymphoma, leukemia, or gastric cancer. In one embodiment, the subject has pancreatic cancer.

Another embodiment is directed to a composition comprising genicitabine and a polynucleotide construct encoding for HuR. In this embodiment, the construct may comprise SEQ ID NO: 11.

Another aspect of the invention is directed to a method of assessing the efficacy of cytarabine (Ara-C) treatment of cancer in a subject comprising examining a biological sample from the subject, measuring the expression level and/or activity level of Human Antigen R (HuR) in the sample, and identifying the subject as resistant to or responsive to Ara-C treatment, wherein an elevated level of HuR in the cells relative to normal cells or cells of a non-responding subject indicates that the subject is responsive to Ara-C treatment. In this embodiment, the HuR may be cytoplasmic HuR. In one embodiment, an elevated expression level or activity level of HuR is correlated with responsiveness to Ara-C treatment. In another embodiment, a negative expression or activity level of HuR relative to normal cells or cells of a non-responding subject is correlated with resistance to Ara-C treatment. In one embodiment, the biological sample is a tumor sample from a biopsy or surgical resection. In another embodiment, the level of expression and/or activity of HuR is measured by immunohistochemistry, immunoprecipitation, or real time PCR. The subject may have pancreatic cancer, small cell lung cancer, colorectal, head and neck cancer, ovarian cancer, melanoma, renal cell carcinoma, non-small cell lung cancer, bladder cancer, ooesophageal cancer, lymphoma, leukemia, or gastric cancer. Another embodiment includes where an elevated level of cytoplasmic HuR expression compared to negative cytoplasmic HuR expression levels is correlated with an increased therapeutic efficacy of Ara-C.

Another embodiment of the invention includes a method of enhancing the efficacy of cytarabine (Ara-C) treatment of a cancer subject comprising increasing the expression level of HuR in said subject. In this embodiment, the HuR may be cytoplasmic HuR. The invention includes the co-administration of Ara-C and a polynucleotide construct encoding for HuR. The invention includes, in another embodiment, where the subject is first administered polynucleotide construct encoding for HuR and then Ara-C is administered. Another embodiment includes where the subject is first administered Ara-C and then administered a polynucleotide construct encoding for HuR. In another embodiment, the subject has pancreatic cancer, small cell lung cancer, colorectal, head and neck cancer, ovarian cancer, melanoma, renal cell carcinoma, non-small cell lung cancer, bladder cancer, ooesophageal cancer, lymphoma, leukemia, or gastric cancer.

The present invention further includes a composition comprising cytarabine and a polynucleotide construct encoding for HuR. Another embodiment includes where the construct comprises SEQ ID NO: 11.

Disclosed herein in one aspect are compositions comprising a polynucleotide encoding an RNA binding protein, such as HuR, and a nucleoside analog such as, for example, gemcitabine or Ara-C. It is understood and herein contemplated that the disclosed compositions can be used to treat cancers including, but not limited to, pancreatic cancer, ovarian cancer, breast cancer, non-small cell lung cancer, and liver cancer.

Disclosed herein in another aspect are compositions for increasing the efficacy of gemcitabine or other nucleoside analog treatments. Also disclosed are methods and kits for increasing the efficacy of a nucleoside analog treatment of a cancer or other disease.

Also disclosed herein are kits and methods for assessing the suitability of a nucleoside analog treatment (such as, for example, gemcitabine) in vitro and in a subject with a cancer.

### 4. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the characterization of HuR-overexpressing pancreatic cancer cell lines. FIG. 1A shows an immunoblot analysis of HuR expression in lysates from MiaPaCa2 (Mia.HuR and Mia.EV) and Hs766T (Hs766t. HuR and Hs766t) cells. Fast Green staining confirmed the equality of protein loading. FIG. 1B shows the use of immunofluorescence to detect HuR and nuclei (DAPI). FIG. 1C shows Mia.HuR and Mia.EV cell proliferation rates, as determined by direct cell counts. FIG. 1D shows that cell survival was measured by PicoGreen after incubation of cells for 5-7 days with the indicated compounds. Data show the means (and S.E.M.) from 3 measurements in a single experiment; each experiment is representative of at least three individual experiments. ▴, Mia HuR cells; ▪, Mia.EV cells.

FIG. 2 shows that stable expression of HuR renders cells hypersensitive to the nucleoside analogs GEM and Ara-C*. FIG. 2A shows that the survival of MiaPaCa2, Hs766t, and PL5 cell lines was measured by the PicoGreen assay after 5-7 days of incubation with the indicated GEM doses, Graphs represent single experiments (S.E.M.); each experiment is representative of >three individual experiments. ▴, HuR expressing cells; ▪, control cells. FIG. 2B shows crystal violet-stained flasks of Mia.HUR and Mia.EV cultures after GEM treatment (0.1 μM, 7 days). FIG. 2C shows the sensitivity of MiaPaCa2 cells to Ara-C treatment was measured as explained in panel (A). FIG. 2D shows FACS analysis of cells treated with GEM (0.03 μM) for 48 h, depicting the percentages of cells in G1, S, and G2/M compartments (left). Measurement of apoptotic fractions in cultures treated as explained in panel (Right).

FIG. 3 shows that HuR associates with dCK mRNA and promotes dCK protein expression in MiaPaCa2 cells. FIG. 3A shows a Western blot analysis of HuR levels in whole-cell and cytoplasmic lysates after treatment of MiaPaCa2 cells with GEM (1 μM) for the indicated times (left). Immunofluorescence analysis of HuR levels and localization in cells treated with 4 μM GEM for 24 h; nuclei were distinguished by staining with DAN (right). FIG. 3B shows a biotin pulldown analysis of HuR RNP complexes. Cytoplasmic extracts were incubated with biotinylated transcripts spanning the DCK or GAPDH 3′ UTRs. The association of HuR with biotinylated RNAs was tested by Western blot analysis. Positive control: HuR cytoplasmic lysate. Negative controls: ‘Probe only’ lanes contain only biotinylated RNAs that were not incubated with protein lysates. Shown is a representative blot (right). HuR binding to dCK mRNA was tested by RNP IP analysis in MiaPaCa2 cells treated with GEM for the times indicated; GEM mRNA levels in HuR and IgG IP samples were first normalized to GAPDH mRNA levels in the same IP reactions, and plotted as fold enrichment in dCK mRNA in HuR IP compared with IgG IP. Data show the means and standard deviation from 3 independent experiments (left). FIG. 3C shows dCK mRNA levels were measured in cells that were left untransfected (left) or were transfected with either a control siRNA or HuR siRNA(7) and tested 48 h later (right). FIG. 31) shows western blot analysis of HuR, dCK, and α-Tubulin in cells expressing normal or silenced HuR levels (left). Immunofluorescence analysis of dCK levels (indicated by the arrow) and localization in cells expressing normal or elevated HuR levels; nuclei were visualized by staining with DAPI (right).

FIG. 4 shows that HuR cytoplasmic expression correlates with GEM response in pancreatic cancer patients. FIG. 4A shows arrows to indicate primarily nuclear staining of HuR in normal pancreas (200×). FIG. 4B shows arrows to indicate high cytoplasmic expression in PDA specimen (200×). FIG. 4C shows a Kaplan-Meier plot of overall survival among patients receiving GEM (n=32) stratified by HuR levels. The curves are significantly different (p=0.0036 by log-rank).

FIG. 5 shows that nanoparticle delivery of DT-A DNA to MSLN+ cells inhibits protein synthesis dramatically. FIG. 5A shows luciferase activity measured in MSLN+ cell lines, Hs766T (left panel) and CAPAN1 (right panel) 24 h post-transfection with (MSLN/XX+CAG/Luc) DNA and (MSLN/DT-A+CAG/Luc DNA. FIG. 5B shows cell survival assays of MSLN+ pancreatic cancer cells, Hs766T, and the MSLN− pancreatic cancer cell line, PL.5. Total number of viable cells was enumerated manually 6 days post-delivery by trypan blue staining. Percent viability was determined by calculating total number of viable cells compared to untreated cultures. Experiments were performed in duplicate with two measurements made for each well (error bars represent SEM).

### 5. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### 5.1. DEFINITIONS

As used in the specification and the appended claims, the singular forms “a”, “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

As used herein, HuR refers to a polynucleotide sequence encoding all or a portion of HuR, an RNA binding protein. The polynucleotide sequence may be incorporated in any of the vectors or DNA constructs taught herein or known to those skilled in the art, and may be delivered to the subject or to particular cells or tissues using the polynucleotide delivery methods taught herein or known by those skilled in the art. In particular instances, as are shown by the context of the statement, HuR may refer to a protein or protein fragment. Antibodies to HuR may be directed to the protein HuR or to the polynucleotide encoding HuR, as noted in the context of the statement.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### 5.2. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular HuR, MSLN, dCK, or DT-A is disclosed and discussed and a number of modifications that can be made to a number of molecules including the HuR, MSLN, dCK, or DT-A are discussed, specifically contemplated is each and every combination and permutation of HuR, MSLN, dCK, or DT-A and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Disclosed herein, in one aspect, are compositions comprising a nucleoside analog and a polynucleotide construct encoding for an mRNA binding protein. It is understood and herein contemplated that the disclosed compositions can be used for many therapeutic purposes including, but not limited to, the treatment of cancer.

Pancreatic ductal adenocarcinoma (PDA) is the fourth leading cause of cancer-related deaths in the United States. Currently, two therapeutic options that provide the best clinical benefit are surgical resection and chemotherapy regimens that include gemcitabine (GEM) (2′,2′-difluorodeoxycytidine, a nucleoside analog).

### Gemcitabine

Gemcitabine (4-amino-1-[3,3-difluoro-4-hydroxy-5-(hydroxymethyl)tetrahydrofuran-2-yl]-1H-pyrimidin-2-one) is an analog of deoxycytidine where the 2′ carbons are replaced with fluorine. Gemcitabine is a prodrug that requires cellular uptake and metabolism to generate the active metabolites, gemcitabine di- and triphosphates, which then in turn inhibit DNA chain elongation and cause cellular death. During DNA replication occurring in the S phase of the cell cycle, gemcitabine replaces cytidine resulting in cell cycle arrest and apoptosis. Because gemcitabine is a diphosphate molecule, it also inhibits ribonucleotide reductase which results in the decreased production of cytidine tri-phosphate. Typically, in the chemotherapeutic setting, gemcitabine is administered via intravenous infusion at a dose of between 1000-1500 mg/m² over a thirty minute period. Thus, for example, the present invention includes compositions comprising a nucleoside analog, and a polynucleotide construct encoding for an mRNA binding protein, wherein the nucleoside analog is gemcitabine.

As noted above, disclosed herein are compositions comprising a nucleoside analog and a polynucleotide construct encoding for an mRNA binding protein, wherein, in one embodiment, the nucleoside analog is gemcitabine. However, it is understood and herein disclosed that the mRNA binding protein and nucleoside analog comprising compositions can comprise any nucleoside analog known. In one aspect, the nucleoside analog can be a nucleoside analog that is used as a chemotherapeutic. For example, it is contemplated herein that the nucleoside analog can be Gemcitabine (GEM), Cytarabine (Ara-C), clofarabine, BCH-4556, troxacitabine, Vidarabine, Zidovudine (also known as Azidothymidine), and 1-(2-deoxy-2-fluoro-4-thio-β-D-arabinofuranosyl)cytosine (4′-thio-FAC). Therefore, this invention includes compositions comprising a nucleoside analog and a polynucleotide construct encoding for an mRNA binding protein, wherein the nucleoside analog is a nucleoside analog other than gemcitabine. For example, disclosed herein are compositions wherein the nucleoside analog is Ara-C.

For over ten years, GEM has been the reference drug for the treatment of pancreatic ductal adenocarcinoma (Burris H A, et al. J Clin Oncol 1997; 15:2403-13). GEM is also utilized to treat other malignancies including non-small cell lung, breast, gastric, and ovarian cancers. GEM utilizes the same key metabolic enzyme for activation within the cell, deoxycytidine kinase (dCK), as does a previously developed and related nucleoside analog cytarabine (Ara-C) (Li Z R, et al. Cancer Treat Rep 1983; 67:547-54). dCK phosphorylates the prodrug, GEM, generating the active metabolites gemcitabine di- and triphosphates that inhibit DNA chain elongation and cause cellular death (Sebastiani V, et al. Clin Cancer Res 2006; 12:2492-7). The levels of dCK correlate with overkill patient survival following GEM-based therapy in PDA specimens (p=0.0425) (Sehastiani V, et al. Clin Cancer Res 2006; 12:2492-7), Herein a group of 40 resected PDA patients was analyzed, of which 30 received GEM, alone or in combination with radiation therapy (4 patients). The median overall survival for patients on GEM was 619 days, with 18 deaths out of the 30 patients who received GEM. However, it has been found that a significant difference was observed in the survival between low and high cytoplasmic Human antigen R (HuR) levels (p=0.025). (HuR is an mRNA binding protein). Kaplan-Meier plot of overall survival among patients receiving GEM (n=32), stratified by HuR levels. The curves are significantly different (p=0.0036 by log-rank). A 7-fold increase in risk of death was seen in patients with low HuR levels compared to high HuR levels among patients receiving GEM.

The present invention contemplates increasing the level of an RNA binding protein such as HuR in subjects receiving a nucleoside analog to increase the effectiveness of a nucleoside analog such as GEM or Ara-C and decreasing the risk of death. In accordance with this embodiment, the level of an RNA binding protein such as HUR is increased through prior or concurrent administration of an RNA binding protein such as HuR or in a composition comprising an RNA binding protein such as HuR and a nucleoside analog such as GEM or Ara-C. The RNA binding protein may be administered in a nucleotide construct encoding for the protein itself.

HuR

HuR (also known as Hu antigen R, ELAVI) is part of the embryonic lethal, abnormal vision, Drosophila-like, mRNA stability protein family that has been shown to have implications in the tumorigenesis process in a number of tumor systems. Functionally, HuR is a protein that stabilizes specific mRNA transcripts based on the sequences embedded in the 3′ and 5′ untranslated regions. HuR is primarily nuclear but can shuttle and stabilize transcripts to the cytoplasm. HuR can shuttle to the cytoplasm when cells are treated with certain drugs, in theory stabilizing specific transcripts in response to stress. Based on previous work, HuR has been shown to post-transcriptionally regulate p21, p27, p53, BCL-2 and a number of other transcripts that have been linked to tumorigenesis and a number of signaling pathways.

Data is herein presented relating to HuR expression in pancreatic tumors. HuR expression levels in pancreatic tumors correlated with patient overall survival for patients receiving gemcitabine-based therapy. Functional aspects of HuR expression were studied in pancreatic cancer cells. The studies revealed that overexpression of HuR in multiple pancreatic cancer cell lines make the cells hypersensitive to nucleoside analogs, gemcitabine and Ara-C.

In accordance with the present invention, one embodiment is directed to the method of increasing the level of HuR in a subject. This method is directed to increasing the expression level and/or the activity level of HuR in the cancer subject. These levels may be measured in any known manner, including but not limited to, immunohistochemistry, immunoprecipitation, real time PCR using a probe specific to HuR, any PCR-based assay, any ELISA-based assay, any protein-based assay, such as mass spectrometry, and in situ hybridization, for example. The levels of HuR may be bulk or total HuR or specific to any part of the cell, where the HuR is normally associated, such as the cytoplasm, nucleus, and cytosol. In another embodiment of the invention, the levels of HuR are measured from the cytoplasm. In the case where cytoplasmic HuR is measured, one may extract cytoplasmic extract, immunoprecipitate the HuR using an HuR antibody, and perform an immunoblot.

The expression/activity levels of HuR in a subject is measured and identified as “elevated” or “negative” in view of levels of HuR in the cells relative to normal cells or cells of a non-responding subject. “Normal cells,” according to the invention, are considered to be cells of a subject that does not have cancer. A “non-responding subject” is defined as a subject that either does not react to or is resistant to the cancer-inhibiting or cancer-treating activity of the nucleoside analog, such as gemcitabine or Ara-C. When measuring levels of the cancer subject and the non-responding subject, the same nucleoside analog should be used by both subjects to determine the HuR levels. In accordance with the present invention, an “elevated” level of HuR is defined as expression/activity of HuR that is higher relative to normal cells or a non-responding subject. A “negative” level of HuR is defined as an expression/activity level that is equal to or less than the level of normal cells or cells of a non-responding subject. In another embodiment, when measuring cytoplasmic HuR, the expression/activity levels of HuR in a subject may be measured and identified as either positive or absent. Therefore, identifying whether or not a subject has an elevated or negative expression or activity level of HuR relative to normal cells or a non-responding subject may be based on information attained by a person practicing the invention provided that the measurements of the cancer subject and the normal cells or cells of a non-responding subject are taken by the same method.

In accordance with the invention, a subject is responsive to nucleoside analogs, such as gemcitabine or Ara-C, if they have elevated levels of HuR relative to the level of normal cells or cells of a non-responding treatment. In this embodiment, the elevated levels may be overexpression (that is, elevated expression over normal cells or cells of non-responding subjects) and/or increased activity of HuR. On the other hand, a subject is considered resistant to nucleoside analogs, such as gemcitabine or Ara-C, if they exhibit negative levels of HuR.

Another embodiment of the invention is directed to a method of enhancing the efficacy of a nucleoside analog treatment of a cancer subject comprising increasing the expression level of HuR in said subject. In this embodiment, a polynucleotide construct encoding for HuR may be delivered to the subject. This construct may be delivered either solely to the HuR, in combination with the nucleoside analog, or before or after the nucleoside analog is delivered. Therefore, in one embodiment, the subject is co-administered gemcitabine or Ara-C and a polynucleotide construct encoding for HuR. In another embodiment, the subject is first administered a polynucleotide construct encoding for HuR and then gemcitabine or Ara-C is administered. In yet another embodiment, the subject is first administered gemcitabine or Ara-C and then administered a polynucleotide construct encoding for HuR. It is possible that the HuR may further be delivered from the nucleus to the cytoplasm to further enhance gemcitabine or Ara-C efficacy. In this manner, a molecule or agent known to be capable of moving HuR from the nucleus to the cytoplasm may be administered along with the construct.

The present invention is further directed to compositions comprising a nucleoside analog and a polynucleotide construct encoding for an mRNA binding protein, wherein the mRNA binding protein is a human embryonic lethal, abnormal vision Drosophila-like (Hu/ELAV) mRNA binding protein such as Human antigen R (HuR). For example, in one embodiment of the invention, compositions comprise a nucleoside analog and a polynucleotide construct encoding for an mRNA binding protein wherein the nucleoside analog is gemcitabine or Ara-C, and the mRNA binding protein is HuR.

Though not wishing to be bound by any particular theory, it is believed that HuR stabilizes the key metabolic enzyme of gemcitabine, deoxycytidine kinase (dCK). Due to the effect that dCK has on gemcitabine, it is understood that the effectiveness of the disclosed compositions in treating cancer can be enhanced through an increase in dCK activity as well as an increase in the activity of transcripts that work in concert with dCK. It is further recognized that an increase in dCK activity alone does not enhance the efficacy of gemcitabine. Accordingly, in one embodiment of the invention, compositions comprise a nucleoside analog, a polynucleotide construct encoding for an mRNA binding protein, and dCK. In another embodiment, the present invention includes compositions comprising a nucleoside analog and an mRNA binding protein, wherein the nucleoside analog is gemcitabine, wherein the mRNA binding protein is HuR, and further comprising polynucleotides encoding dCK or dCK protein.

The compositions comprising a nucleoside analog and a polynucleotide construct encoding for an mRNA binding protein and optionally a polynucleotide construct encoding for dCK can be used for many applications including but not limited to use as a chemotherapeutic to treat a cancer. Moreover, it is understood that there are alternative compositions that can achieve the same effect, lilt is further understood that it may be desirable to provide additional therapeutics to enhance the effectiveness of the compositions disclosed herein. For example, the disclosed compositions can further comprise additional chemotherapeutics or toxic moieties, which can kill targeted molecules. For example, in one embodiment, compositions of the present invention comprise a polynucleotide construct encoding for an mRNA binding protein and a nucleoside analog further comprising a toxin. It is understood and herein contemplated that there are many known toxins that may be used in the disclosed compositions including but not limited to Diphtheria toxin (DT-A). Ricin toxin, Botulinum toxin, Vibrio toxin, and Pertussis toxin. Thus, in one embodiment, the invention includes compositions comprising a polynucleotide construct encoding HuR, GEM, and a polynucleotide construct encoding DT-A. In this embodiment, the polynucleotides may be found in one or more constructs. In another embodiment, the invention includes compositions comprising a polynucleotide construct encoding HuR, GEM, a polynucleotide construct encoding DT-A, and a polynucleotide construct encoding dCK. In this embodiment, the polynucleotides may be found in one or mere constructs.

In an alternative, embodiment, the toxins may be administered as a protein, peptide, or nucleic acid. In this manner, the toxins may be co-administered with other compositions of the invention, including a composition comprising a polynucleotide construct encoding HuR and GEM or Ara-C. These toxins and the compositions may alternatively be administered sequentially.

Diphtheria Toxin-A.

DT-A is a naturally occurring toxin produced by the bacterium Corynebacterium diphtheriae. DT-A encoding DNA has been cloned and the mechanism of action of DT-A is well understood. DT-A is so potent that a single molecule can kill a eukaryotic cell. Prostate and ovarian cancer in vitro and in vivo studies have used DT-A with some success.

In nature, the secreted DT protein is composed of an A and a B chain. The B chain effectively delivers the A chain (DT-A), the toxin, into the cell. Once inside the cell, the DT protein is enzymatically cleaved. The B chain degrades and the DT-A chain (i.e., the toxin) inhibits protein synthesis by catalyzing the ADP-ribosylation of EF2 elongation factor. For use as a therapeutic, only the DT-A sequence encoding the toxin is utilized, nut the coding sequence for the B subunit. Because DNA constructs lack the B subunit, if the toxin is released from dying cells, it is incapable of entering neighboring cells, making this strategy more desirable and specific for targeting cancer cells, particularly pancreatic ductal adenocarcinoma cancer cells.

Delivery of the Compositions to Cells

There are a number of compositions and methods which can be used to deliver nucleic acids to cells, either in vitro or in vivo. These methods and coniposifions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are contemplated herein. Such methods are well known in the art and readily adaptable for use with the compositions and methods described herein. In certain cases, the methods can be modified to specifically function with large DNA molecules. Further, these methods can be used to target certain diseases and cell populations by using the targeting characteristics of the carrier.

5.2.1.1. Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus.

As used herein, plasmid or viral vectors are agents that transport the disclosed nucleic acids, such as an RNA binding protein (e.g., HuR), a cancer specific promoter (e.g., MSLN) or a toxin (e.g., DT-A) into the cell without degradation and include a promoter yielding expression of the gene in the cells into which it is delivered. In some embodiments the vectors or promoters are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. An embodiment is a viral vector which has been engineered so as to suppress the immune response of the host organism, elicited by the viral antigens. Vectors of this type will carry coding regions for Interleukin 8 or 10.

Viral vectors can have higher transaction (ability to introduce genes) abilities than chemical or physical methods to introduce genes into cells. Typically, viral vectors contain nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it a nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication and packaging of the replicated virus. Typically a retroviral genome contains the gag, pal, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of one to many genes depending on the size of each transcript. Either positive or negative selectable markers may be included along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

Adenoviral Vectors

The construction of replication-defective adenoviruses has been described. The benefit of the use of these viruses as vectors is that they, the vectors, are limited in the extent to which the vectors can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites. Recombinant adenoviruses achieve gene transduction by binding to specific cell surface receptors, after which the virus is internalized by receptor-mediated endocytosis, in the same manner as wild type or replication-defective adenovirus.

A viral vector can be one based on an adenovirus which has had the E1 gene removed and these virons are generated in a cell line such as the human 293 cell line. In another embodiment both the E1 and E3 genes are removed from the adenovirus genome.

Adeno-Associated Viral Vectors

Another type of viral vector is based on an adeno-associated virus (AAV). This defective parvovirus can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Large Payload Viral Vectors

Molecular genetic experiments with large human herpesviruses have provided a means whereby large heterologous DNA fragments can be cloned, propagated and established in cells permissive for infection with herpesviruses. These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

5.2.1.2. Non-Nucleic Acid Based Systems

Nanoparticle Delivery of DNA

A promising and already well-tested non-viral vector for delivering DNA is a class of cationic polymers, poly(β-amino ester)s (PBAE), which hind and condense DNA to form nanoparticles. A wide variety of polymers have been tested in vitro and in vivo for efficacy. Thousands of PBAE formulations were tested for in vitro transfection efficiency and cytotoxicity previously and the best-performing formulations were then tested in mice. The PBAE, C32, was used in studies to deliver diphtheria toxin DNA to prostate tumors, successfully reducing their size. Subsequently, it was discovered that minor modifications to the ends of PBAEs changed their ability to deliver DNA more effectively. Specifically, a modification to the ends of C32 significantly enhanced its ability to deliver DNA to multiple organs. Modified C32, a formulation called C32-117, was used herein.

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed nanoparticles or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. For example, administration of a composition comprising a compound and a cationic liposome can be administered to the blood afferent to a target organ or inhaled into the respiratory tract to target cells of the respiratory tract. A composition may be administered as a component of a microcapsule that can be targeted to specific cell types, such as macrophages, or where the diffusion of the composition or delivery of the composition from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo may be used. The following references teach targeting specific proteins to tumor tissue (Hughes et al., Cancer Research, 49:6214-6220, (1989); and Litzinger and Huang; Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycled to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed.

Nucleic acids that are delivered to cells which are to be integrated into the host cell genome, typically contain integration sequences. These sequences are often viral related sequences, particularly when viral based systems are used. These vital intergration systems can also be incorporated into nucleic acids which are to be delivered using a non-nucleic acid based system of delivery, such as a liposome, so that the nucleic acid contained in the delivery system can be come integrated into the host genome.

Other general techniques for integration into the host genome include, for example, systems designed to promote homologous recombination with the host genome. These systems typically rely on sequences flanking the nucleic acid to be expressed that have enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

5.2.1.3. In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication. The immediate early promoter of the human cytomegalovirus is conveniently obtained as a HindIII E restriction fragment. Of course, promoters from the host cell or related species also are useful herein.

Enhancer generally refers to it sequence of DNA that functions at no fixed distance from the transcription start site and can be either 5′ or 3′ to the transcription unit. Furthermore, enhancers can be within an intron as well as within the coding sequence itself. They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the snRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. Homologous polyadenylation signals may be used in the transgene constructs. In certain transcription units, the polyadenylation region is derived from the SV40 early polyadenylation signal and consists of about 400 bases. The transcribed units may contain other standard sequences alone or in combination with the above sequences improve expression from, or stability of, the construct.

### Markers

The viral vectors can include nucleic acid sequence encoding a marker product. This marker product is used to determine if the gene has been delivered to the cell and once delivered is being expressed. Marker genes include the E. Coli lacZ gene, which encodes β-galactosidase, and green fluorescent protein.

MSLN

Mesothelin (MSLN) is a 69 kDa protein that is cleaved into a roughly 40 kDa membrane bound protein and a soluble 31 kDa fragment termed the megakaryocyte potentiating factor. Mesothelin is typically expressed in normal human tissues such as the mesothelial cells lining the pleura, pericardium and peritoneum. The membrane bound protein is glycosylphosphatidyl inositol (GPI)-anchored. MSLN is overexpressed in a variety of cancers including but not limited to pancreatic cancer, lung cancer, and ovarian cancer. Therefore, it is understood and herein contemplated that a vector comprising the MSLN promoter operably linked to a nucleic acid encoding a protein will limit expression of the protein to cancerous tissue, for example, ovarian cancer or pancreatic cancer tissue. For example, the compositions disclosed herein comprise DNA constructs that express mRNA binding proteins or toxin moieties driven by the MSLN promoter. Thus, herein are compositions comprising nucleic acid constructs encoding for an mRNA binding protein and a nucleoside analog wherein the gene for the mRNA binding protein (e.g., HuR) is operably linked to the MSLN promoter. Also disclosed are compositions further comprising nucleic acid constructs encoding a toxin moiety, such as DT-A, wherein the gene for the toxin moiety is operably linked to the MSLN promoter. In a further aspect of the invention, the disclosed compositions may comprise a toxin moiety encoded by a vector operably linked to the MSLN promoter. In still another aspect of the invention, the composition can comprise nucleic acids encoding HuR and DT-A operably linked to the MSLN promoter, a nucleoside analog such as gemcitabine, and a polynucleotide construct encoding for dCK. The genes for HuR, DT-A and dCK may be separately under the control of the MSLN promoter, or one or more are under the control of the MSLN promoter and one or more may be found on one or more nucleotide constructs.

### Cancer Enhancing Transcription Sequence

The Cancer Enhancing Transcription Sequence (CanScript) (CanSCRIPT (1×): CTC CAC CCA CAC ATT CCT GG (SEQ ID NO: 12) CanSCRIPT (2×): CTC CAC CCA CAC ATT CCT GG CTC CAC CCA CAC ATT CCT GG (SEQ ID NO: 13) CanScript (×3): CTC CAC CCA CAC ATT CCT GGCTC CAC CCA CAC ATT CCT GGCTC CAC CCA CAC ATT CCT GG (SEQ ID NO: 14)) of MSLN is a TEF-1 binding site. The CanScript is responsible for enhanced cancer specific transcription. Moreover, three repeats of the CanScript sequence inserted in front of a minimal promoter enhanced cancer specific transcription 30-fold. Accordingly, disclosed herein in one aspect are compositions comprising a polynucleotide construct encoding for an mRNA binding protein gene and a nucleoside analog, wherein the polynucleotide construct encoding for mRNA binding protein gene is operably linked to at least one, two, three, four, or five CanScript sequences. Thus, disclosed herein are compositions comprising a polynucleotide construct encoding for an mRNA binding protein and a nucleoside analog wherein a polynucleotide construct encoding for the mRNA binding protein is operably linked to one or more, two or more, three or more, four or more, five or more Canscript sequences.

### PSCA

Prostate Stem Cell Antigen (PSCA) is a GPI-linked cell surface membrane protein that has been shown to be overexpressed in the majority of pancreatic cancer cells and not in normal pancreatic cells. PSCA has been found to be overexpressed in roughly 50% of precursor lesions of pancreatic cancer (PanINs). It is therefore contemplated herein, that a polynucleotide construct encoding for the mRNA binding protein and/or a polynucleotide construct encoding for toxin moieties and/or deoxycytidine kinases in the disclosed compositions can also be driven by the PSCA promoter. Thus, disclosed herein are compositions comprising an mRNA binding protein gene and a nucleoside analog, wherein a polynucleotide construct encoding for an mRNA binding protein is operably linked to the PSCA promoter. In a further aspect of the invention, disclosed herein are compositions comprising a polynucleotide construct encoding for HuR and gemcitabine wherein HuR is encoded on a nucleic acid vector, wherein the nucleic acid encoding HuR is operably linked to the PSCA promoter.

It is understood and herein contemplated that the disclosed compositions can be used with any tissue specific promoter. One of skill in the art can determine the appropriate promoter given the tissue to be targeted. Examples of other tissue specific promoters that can be used in the disclosed compositions include but are not limited to MSLN promoter, PSCA promoter, prostate specific antigen (PSA) promoter, ARR2PB, Pancreatic duodenal homeobox 1 (PDX) promoter, probasin (PB) promoter, and prostate specific antigen enhancer promoter (PSE-BC).

It is a further aspect of the invention that in addition to targeted expression/delivery of the disclosed compositions, conditional expression may also be desired such as an inducible promoter. Thus disclosed herein in one aspect are compositions wherein the mRNA binding protein gene and/or the toxin moiety gene is under control of an inducible expression system. Those of skill in the art are intimately familiar with available conditional expression systems and the advantages of each.

Accordingly, those of skill in the art can choose the appropriate expression system given the expression control desired and the tissue type in which expression will occur. Inducible expression systems can include, but are not limited to the Cre-lox system, Flp recombinase, and tetracycline responsive promoters. Any recombinase system can be used. The Cre recombinase system which when used will execute a site-specific recombination event at loxP sites. A gene that is flanked by the loxP sites, flexed, is excised from the transcript. Control of the recombination event, via the Cre Recombinase, can be constitutive or inducible, as well as ubiquitous or tissue specific, depending on the promoter used to control Cre expression.

### Combination Therapies

It is understood and herein contemplated that the disclosed compositions can be used in conjunction with other compositions known treatments for cancer including but not limited to radiation therapy (including but not limited to gamma and UV irradiation) and chemotherapeutics (e.g., XELODA® (Capecitabine). It is further understood that the disclosed compositions can be administered in conjunction with antibiotics, including but not limited to Amikacin, Neomycin, Penicillin, Amoxicillin, Ampicillin, Bacitracin, Tetracycline, Streptomycin, Gentamicin, and Kanamycin. It is understood that such compositions may increase the efficacy of a treatment through an additional mechanism of action against a cancer or by activating HuR and thus having an adjuvant effect on the compositions disclosed herein. For example, disclosed herein is the use of neomycin, irradiation, infrared light to further activate HuR and increase sensitivity to gemcitabine. Thus, for example disclosed herein are methods of treating a cancer comprising administering to a subject a nucleoside analog, an mRNA binding protein, an antibiotic, and/or irradiation. It is understood and herein contemplated that the mRNA binding protein (e.g., HuR) can be encoded on a vector and provided before, concurrent with, after or in the same composition with the nucleoside analog. It is further contemplated that the irradiation or antibiotic (e.g., neomycin) can be administered before, concurrent with, after or in the same composition with the nucleoside analog and/or the mRNA binding protein. Thus, also disclosed are compositions comprising a nucleoside analog, an antibiotic, and polynucleotide construct encoding an mRNA binding protein. Further disclosed are compositions comprising a nucleoside analog, an antibiotic, and polynucleotide construct encoding an mRNA binding protein, wherein the nucleoside analog is gemcitabine, the mRNA binding protein is HuR, and the antibiotic is neomycin.

Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector or protein, without causing any undesirable biological effects or interacting in a deleterious manner with any of the other components of the pharmaceutical composition in which it is contained. The carrier would naturally be selected to minimize any degradation of the active components and to minimize any adverse side effects in the subject, as would be well known to one of skill in the art.

The compositions may be administered orally, parenterally (e.g. intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the flares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

Pharmaceutically Acceptable Carriers

The compositions, can be used therapeutically in combination with a pharmaceutically acceptable carrier including, for example, sterile water. Suitable carriers and their formulations are described in Remington: The Science end Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carriers include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the composition, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical compositions may include carriers, thickeners, diluents, buffers, preservatives, surface active agents and the like in addition to the disclosed compositions. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed compositions can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amities and substituted ethanolamines.

Therapeutic Uses

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products.

The disclosed compositions and methods can also be used for example as tools to isolate and test new drug candidates for a variety of cancer related diseases.

Method of Treating Cancer

The disclosed compositions can be used to treat any disease where uncontrolled cellular proliferation occurs such as cancers. “Treatment,” “treat,” or “treating” mean a method of reducing the effects of a disease or condition. Treatment can also refer to a method of reducing the disease or condition itself rather than just the symptoms. The treatment can be any reduction from native levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression. For example, a disclosed method for reducing the effects of pancreatic cancer is considered to be a treatment if there is a 10% reduction in one or more symptoms of the disease in a subject with the disease when compared to native levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. For example, prolonged survival is understood to be included within the understanding of the term “treatment.” Accordingly, a patient is treated with a composition if after administration of the composition, the patients survival increases 10, 20, 30, 40, 50, 60, 70, 80, 90, 100%, or any amount of survival in between relative to control subjects not receiving the treatment.

Thus disclosed in one aspect are methods of treating a cancer in a subject comprising administering to the subject the compositions disclosed herein.

A representative but non-limiting list of cancers that the disclosed compositions can be used to treat is the following: lymphoma, B cell lymphoma, T cell lymphoma, mycosis fungoides, Hodgkin's Disease, leukemias, myeloid leukemia, multiple myeloma, histicytic malignant proliferations, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, malignant melanoma, carcinomas and adenocarcinomas, squamous cell carcinomas of the mouth, throat, larynx, and lung, metastatic cancers, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, CNS and peripheral nervous system tumors, PNETs, sarcomas, germ cell and stromal tumors, hematopoietic cancers; testicular cancer; malignant neoplasms, colon and rectal cancers, or pancreatic ductal adenocarcinoma.

Compositions disclosed herein may also be used for the treatment of precancer conditions such as cervical and anal dysplasias, other dysplasias, severe dysplasias, hyperplasias, atypical hyperplasias, and neoplasias in situ. Thus, for example, herein disclosed are methods of treating pancreatic ductal adenocarcinoma, prostate, ovarian, breast, or lung cancer in a subject comprising the compositions disclosed herein. In one aspect, the disclosed compositions can comprise a nucleoside analog and a polynucleotide construct encoding for an mRNA binding protein. Thus, in one aspect, disclosed herein are methods of treating a cancer in a subject comprising administering to the subject a composition comprising a nucleoside analog and a polynucleotide construct encoding for an mRNA binding protein in a further aspect, disclosed herein are methods of treating a cancer in a subject comprising administering to the subject a composition comprising a nucleoside analog and a polynucleotide construct encoding for an mRNA binding protein, wherein the nucleoside analog is gemcitabine, wherein the mRNA binding protein is HuR, and wherein the cancer is prostate cancer. It is understood and herein contemplated that any of the compositions disclosed herein can be used to treat a cancer. Thus, disclosed herein are methods of treating a cancer in a subject comprising administering to the subject a composition comprising one or more of a polynucleotide construct encoding for an mRNA binding protein, a nucleoside analog, a polynucleotide construct encoding for dCK, and a polynucleotide construct encoding for a toxin moiety. Also disclosed herein are methods of treating a cancer in a subject comprising administering to the subject a composition comprising a polynucleotide construct encoding for two or more of a mRNA binding protein, a nucleoside analog, a polynucleotide construct encoding for dCK, and a polynucleotide construct encoding for a toxin moiety. Herein are methods of treating a cancer in a subject comprising administering to the subject a composition comprising three or more of a polynucleotide construct encoding for a mRNA binding protein, a nucleoside analog, a polynucleotide construct encoding for dCK, and a polynucleotide construct encoding for a toxin moiety. It is understood that these polynucleotides may be found in one or more DNA constricts comprising the polynucleotide sequences. It is understood and herein contemplated that for these treatment methods, any of the disclosed mRNA binding proteins, toxin moieties or nucleoside analogs or nucleic acids encoding them can be used. Thus, specifically contemplated herein are methods of treating cancer comprising administering a composition comprising a polynucleotide construct encoding for HuR, and gemcitabine. Also disclosed are compositions further comprising a polynucleotide construct encoding for dCK and/or a polynucleotide construct encoding for DT-A. In another aspect, disclosed herein are methods of treating cancer comprising administering a composition comprising a polynucleotide construct encoding for HuR and Ara-C. It is further understood that the disclosed treatment methods can utilize the disclosed compositions delivered by any means disclosed herein. For example, disclosed herein are methods of treating a cancer in a subject comprising administering to the subject a composition comprising gemcitabine and HuR, wherein HuR is a nucleic acid encoded on a vector operably linked to the MSLN promoter.

It is further understood that rather than the administration of a single composition, the disclosed treatment methods can be achieved through the separate administration of at least a polynucleotide construct encoding for an mRNA binding protein and a nucleoside analog. Thus, disclosed herein are methods of treating a cancer comprising administering to the subject a polynucleotide construct encoding for an mRNA binding protein and a nucleoside analog. It is understood and herein contemplated that the a polynucleotide construct encoding for mRNA binding protein can be administered prior to, concurrent with, or after the administration of the nucleoside analog. Thus, disclosed herein are methods of treating a cancer in a subject comprising administering to the subject gemcitabine and a polynucleotide construct encoding for HuR, wherein a polynucleotide construct encoding for HuR is administered to the subject prior to the administration of gemcitabine. Also disclosed are methods wherein it polynucleotide construct encoding for HuR is administered concurrently with gemcitabine. It is understood and herein contemplated that the polynucleotide construct encoding for the disclosed mRNA binding protein, toxin moiety, dCK, and the nucleoside analog can be delivered in a single formulation, separate formulations or any combination thereof. A nucleotide construct may code for one or more of the genes of interest.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels. Moreover, inhibition can refer to any increase in the survival rate of a subject after administration of the disclosed compositions to the subject relative to controls. Thus, a 10, 20, 30, 40, 50, 60, 70, 80, 90, 100% or any other increase in survival rate indicates that the disease, condition, or other biological parameter is inhibited.

Methods of Assessing Efficacy of a Treatment

A significant difference in the survival of patients was observed between low and high cytoplasmic Human antigen R (HuR) levels (p=0.0036). Specifically, a 7-fold increase in risk of death was seen in patients with low HuR levels compared to high HuR levels among patients receiving GEM. Thus, one method for determining the efficacy of a treatment with gemcitabine or Ara-C in a subject is to measure the levels of HuR in the subject, wherein an increase in the levels of HuR in the subject relative to a control indicates an efficacious treatment. The level of HuR may be determine for any location in the cell in which HuR levels may be assessed. In one embodiment, the HuR levels are determined in the cytoplasm of the cell. Thus, in one embodiment, the invention is directed to methods of assessing the efficacy of gemcitabine treatment of a cancer in a subject comprising obtaining a biological sample, such as a tissue sample, from the subject and measuring the level of cytoplasmic HuR in the cells of the tissue, wherein an elevated level (as described above) or an increase in the cytoplasmic HuR in the cells relative to a control indicates an efficacious treatment. The biological sample can be any sample including tumor samples, such as those from biopsies or surgical resection.

An “increase” can refer to any change that results in a larger amount of a composition, protein, or compound, such as HuR relative to a control. The control is the level of HuR in a normal cell or in a cell of a non-responding subject, as earlier defined. Thus, for example, an increase in the amount in HuR can include but is not limited to a 10%, 20%, 30%, 40%, 50%, 60%, 70%, 80%, 90%, or 100% increase are any amount in between.

A “decrease” can refer to any change that results in a smaller amount of a composition or molecule, such as HuR. Thus, a “decrease” can refer to a reduction in an activity or expression level of a protein, compound, or composition. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed.

It is understood and herein contemplated that there are many methods known in the art that can be used to measure the levels of HuR in a tissue sample. Such methods include, but are not limited to immunoblot, in immunofluorescence, cutting edge matrix assembly (CEMA), and automated quantitative analysis. Thus, disclosed herein, fix example, are methods of assessing the efficacy of gemcitabine treatment of a cancer in a subject comprising obtaining a tissue sample from the subject and measuring the level of cytoplasmic HuR in the cells of the tissue, wherein an increase in the cytoplasmic HuR in the cells relative to a control indicates an efficacious treatment, and wherein the HuR levels are measured by tissue array (e.g. CEMA), immunoblot, or immunofluorescence (e.g. AQUA).

Methods of Assessing the Suitability of a Treatment

In addition to determining the efficacy of a treatment with gemcitabine or Ara-C, it is understood and herein contemplated that the levels of cytoplasmic HuR in a subject can also be used to determine if the subject is a suitable candidate for gemcitabine or Ara-C treatment. Thus, disclosed are methods of assessing the suitability of gemcitabine treatment of a cancer in a subject comprising obtaining a tissue sample from the subject and measuring the level of HuR, such as cytoplasmic HuR, in the cells of the tissue, wherein an increased level of HuR, i.e., cytoplasmic HuR, in the tissue relative to a control indicates that the subject is a suitable candidate for gemcitabine or Ara-C treatment.

It is understood and herein contemplated that there are many methods known in the art that can be used to measure the levels HuR in a tissue sample. Such methods include, but are not limited to immunoblot, immunofluorescence, cutting edge matrix assembly (CEMA), and automated quantitative analysis. Thus, disclosed herein, for example, are methods of assessing the efficacy of gemcitabine treatment of a cancer in a subject comprising obtaining a tissue sample from the subject and measuring the level of cytoplasmic HuR in the cells of the tissue, wherein an increase in the cytoplasmic HuR in the cells relative to a control indicates an efficacious treatment, and wherein the HuR levels are measured by tissue array (e.g., CEMA), immunoblot, or immunofluorescence (e.g., AQUA).

Methods of Increasing the Efficacy

Because levels of HuR are proportionally related to the effectiveness of nucleoside analog treatment of a cancer, it is possible to increase the efficacy of said treatment by increasing the level of HuR. In one embodiment, cytoplasmic levels or HuR are increased. It has been found that greater than 5% of the cells in a tumor exhibited high or elevated or positive cytoplasmic HuR expression. Therefore, it is one embodiment of the invention to enhance efficacy of nucleoside analogs by increasing the percentage of tumor cells in the cancer subject having elevated HuR or positive cytoplasmic HuR expression because these cells are more susceptible to nucleoside analog treatment. In another aspect of the invention, disclosed herein are methods of increasing the efficacy of a composition for treating a cancer in a subject comprising administering to the subject, HuR or a nucleic acid construct encoding HuR. In a further aspect, disclosed herein are methods of increasing the efficacy of a nucleoside analog treatment for a cancer in a subject comprising administering to the subject a polynucleotide construct encoding for HuR. In yet another aspect, disclosed herein are methods of increasing the efficacy of a nucleoside analog treatment for a cancer in a subject comprising administering to the subject a polynucleotide construct encoding for HuR, wherein the nucleoside analog is gemcitabine. Also disclosed are methods of increasing the efficacy of a nucleoside analog treatment of a cancer, wherein the nucleoside analog is Ara-C. It is understood that as with the methods of treating a cancer, a polynucleotide construct encoding for HuR can be administered prior to, concurrent with, or after gemcitabine treatment. Moreover, it is contemplated herein that a polynucleotide construct encoding for HuR can not only be administered concurrent with nucleoside analog treatment, but can be in the same or separate formulation as the nucleoside analog. It is further contemplated herein that a polynucleotide construct encoding for HuR may be delivered to the subject utilizing any of the methods disclosed herein. For example, HuR may be delivered as a nucleic acid encoding HuR on a vector. Moreover, the HuR on the vector can be operably linked to a tissue specific promoter such as the MSLN promoter, PSCA promoter, or probasin promoter. Alternatively, the HuR gene can be operably linked to one or more, two or more, three or more, or four or more CanScript sequences. The HuR gene can also be operably linked to an inducible expression system such as the Cre-Lox system. Moreover, it is contemplated herein that a polynucleotide construct encoding for HuR can be bound by poly (β-amino ester)s (PBAE) to form nanoparticles. In still a further aspect, it is understood and herein contemplated that a polynucleotide construct encoding for HuR can be administered concurrently with a polynucleotide construct encoding for toxin moiety such as DT-A or a polynucleotide construct encoding for a kinase such as dCK. Thus disclosed herein are methods of increasing the efficacy of gemcitabine treatment of a cancer in a subject comprising administering to the subject a polynucleotide construct encoding for HuR.

Kits

Disclosed herein are kits that are drawn to reagents that can be used in practicing the methods disclosed herein. The kits can include any reagent or combination of reagents discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed is a kit for assessing the efficacy of gemcitabine treatment of a cancer in a subject comprising an anti-HuR monoclonal antibody and at least one positive or one negative control tissue sample. Also disclosed are kits for assessing the suitability of gemcitabine treatment for a subject with a cancer comprising an anti-HuR monoclonal antibody and at least one positive and one negative control tissue sample. It is understood that the disclosed kits can be used for numerous applications including but not limited to immunoblot detection, immunofluorescence detection (e.g. AQUA), and tissue array (e.g., CEMA). Thus, the disclosed kits can be modified to be more suitable for each given application. It is further understood that there are numerous means to detect the presence of monoclonal antibody binding. Such methods can include direct detection through the use of a labeled monoclonal antibody or through detection of a secondary antibody which is labeled and which secondary antibody binds to the monoclonal antibody. Examples can include monoclonal antibodies to HuR but can also include antibodies capable a detecting the phosphorylation state of HuR, for example, an antibody which only binds to phosphorylated HuR. Thus, disclosed herein are kits further comprising a secondary antibody that can bind to the monoclonal antibody. Alternatively detection mechanisms include visualization reagents such as horseradish peroxidase. It is further contemplated that said kits can include buffers, blocking reagents, substrates, and retrieval solutions. It is understood that there are many known methods of detection known to those of skill in the art. Specifically contemplated are kits comprising any detection mechanism now known.

Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman, by the homology alignment algorithm of Needleman and Wunsch, by the search for similarity method of Pearson and Lipman, by computerized implementations of these algorithms (GAP, RESTFUL, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

Nucleic Acids

There are a variety of molecules disclosed herein that are nucleic acid based, including for example the nucleic acids that encode, for example HuR, MSLN, or DT-A, or fragments thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell: that the expressed mRNA will typically be made up of A, C, G, and U.

Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety, Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face at a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

Sequences

There are a variety of sequences related to the protein molecules involved in the signaling pathways disclosed herein, for example HuR or MSLN, or any of the nucleic acids disclosed herein for regulating dCK, all of which are encoded by nucleic acids or are nucleic acids. The sequences for the human analogs of these genes, as well as other analogs, and alleles of these genes, and splice variants and other types of variants, are available in a variety of protein and gene databases, including Genbank. Those sequences available at the time of filing this application at Genbank are herein incorporated by reference in their entireties as well as for individual subsequences contained therein. Those of skill in the art understand how to resolve sequence discrepancies and differences and to adjust the compositions and methods relating to a particular sequence to other related sequences. Primers and/or probes can be designed for any given sequence given the information disclosed herein and known in the art.

Protein Variants

As discussed herein there are numerous variants of the HuR or DT-A protein that are known and herein contemplated. In addition, to the known functional HuR, MSLN, dCK, and DT-A strain variants there are derivatives of the HuR, MSLN, dCK, and DT-A proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | AlaA |
| allosoieucine | Alle |
| arginine | ArgR |
| asparagine | AsnN |
| aspartic acid | AspD |
| cysteine | CysC |
| glutamic acid | GluE |
| glutamine | GlnK |
| glycine | GlyG |
| histidine | HisH |
| isolelucine | IleI |
| leucine | LeuL |
| lysine | LysK |
| phenylalanine | PheF |
| proline | ProP |
| pyroglutamic acid | Glu |
| serine | SerS |
| threonine | ThrT |
| tyrosine | TyrY |
| tryptophan | TrpW |
| valine | ValV |

TABLE 2
Amino Acid Substitutions
Original Residue Exemplary Conservative
Substitutions, others are known in the art.
Ala, ser
Arg, lys, gln
Asn, gln; his
asp, glu
Cys, ser
Gln, asn, lys
Glu, asp
Gly, pro
His, asn; gln
Ile, leu; val
Leu, ile; val
Lys, arg; gln;
Met, Leu; ile
Phe, met; leu; tyr
Ser, thr
Thr, ser
Trp, tyr
Tyr, trp; phe
Val, ile; leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO: 10 is set forth in SEQ ID NO: 11. It is also understood that while no amino acid sequence indicates what particular DNA sequence encodes that protein within an organism, where particular variants of a disclosed protein are disclosed herein, the known nucleic acid sequence that encodes that protein in the particular from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology. 12:678-682 (1994) all of which are herein incorporated by reference at least for material related to amino acid analogs).

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

Compositions with Similar Functions

It is understood that the compositions disclosed herein have certain functions, such as binding dCK. Disclosed herein are certain structural requirements for performing the disclosed functions, and it is understood that there are a variety of structures which can perform the same function which are related to the disclosed structures, and that these structures will ultimately achieve the same result.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

The stress-response protein Hu antigen R (HuR) is an RNA-binding protein that regulates gene expression post-transcriptionally. Like other related Hu/elav proteins, HuR harbors three conserved RNA recognition motifs through which it binds to target mRNAs that frequently have AU- or U-rich stretches in the 3′-untranslated regions (UTRs). HuR is predominantly nuclear, but in response to various stimuli, it is mobilized to the cytoplasm, prolongs target mRNA half-life, and can modulate target mRNA translation. Many HuR target mRNAs encode stress-response, immune-response, cell cycle regulatory proteins, oncogenes, and tumor suppressor genes. HuR modulates these transcripts in response to stimuli such as therapeutic agents (i.e. tamoxifen and prostaglandin), nutrient depletion (polyamines, amino acid starvation), heat shock, immune stimuli, short-wavelength UV irradiation, oxidants, and transcriptional inhibitors (actinomycin D).

Transfection of Pancreatic Cancer Cell Lines

HuR cDNA sequence was cloned into the pcDNA 3.1. Zeo vector (Invitrogen) for stable transfection of pancreatic cancer cell lines MiaPaca2, PL-5, and Hs766t. Pooled cells remained under selection media containing Zeocin (Invitrogen) for several months after transfection. Mia.HuR, PL5.HuR, and Hs766t. HuR denote link overexpressing lines; Mia.EV, PL5.EV, and Hs766t denote empty vector or control lines. HuR and control siRNA sequences and transfection conditions were as described.

Immunofluorescence

Cells were plated on LabTek II™ Chamber slides (Fisher Scientific) and fixed in 3% paraformaldehyde (20 min, RT). Cells were washed with PBS and permeabilized using 0.5% Triton X-100/1% normal goat serum (Vector Laboratories) in PBS (15 min). After washes in 1% goat serum/PBS, cells were incubated (1:50 dilution, 1 hr, RT) with mouse anti-HuR (Santa Cruz) or anti-deoxycytidine kinase (dCK, Abnova) primary antibodies. After washes in PBS, cells were incubated for 1 hr with goat anti-mouse secondary antibody (1:400, Alexa Fluor 647, Molecular Probes). Nuclei were stained with DAPI and cells were evaluated under a Zeiss LSM-510 Confocal Laser Microscope.

Drug Sensitivity Assay

Mia.HuR, Mia.EV, Hs766t.HUR, Hs766t, and PL5.HuR, PL5.EV cells were seeded (1000 cells/well) in 96-well plates and treated with Etoposide, 5-Fluorouracil, Cis-platin, Staurosporine, Nocodazole, Colcemid, Ara-C (Sigma), and GEM (Gemzar, Eli-Lilly, Indianapolis, Ind.) for 6-7 days. After treatment, cells were washed with PBS and lysed with 100 μL of water/well; cell viability was quantified by staining of double-stranded DNA with QuantiT™ PicoGreen (Invitrogen) and analyzed with a TECAN SpectraFluor.

Immunoblot

Whole-cell, cytoplasmic, and nuclear lysates were prepared as described (Kuwano Y, et al. Mol Cell Biol 2008; 28:4562-75), and protein was size-fractionated by SDS-PAGE (10% acrylamide). Membranes were blocked for 1 h in 5% Milk/TBS-T and incubated overnight with monoclonal antibodies (Santa Cruz) recognizing HuR, dCK, the cytoplasmic marker α-Tubulin, or the nuclear marker hnRNP. Membranes were washed with TBS-T and incubated with secondary antibodies; and the resulting signals were visualized by chemiluminescence (Millipore). Total protein was visualized with Fast Green (USB).

Cell Cycle Analysis and Apoptosis Assay

Mia.EV and Mia.HuR cell lines were either left untreated or treated with 0.03 μM GEM for 48 h. For cell cycle analysis, cells were then fixed in 100% ethanol, and stained with a propidium iodide solution containing RNAse A (Sigma Aldrich). For apoptosis assays, cells were resuspended at 10₆ cells/mL and incubated in Annexin V and Propidium Iodide, following the manufacturers' protocol (FITC Annexin V, BD Pharmigen). Both assays were analyzed by flow cytometry.

RNA-Binding: Biotin Pulldown and RNP IP Assays

MiaPaCa2 cells were treated with 4 μM GEM and collected 48 hours later. For biotin pulldown analysis (Kuwano Y, et al. Mol Cell Biol 2008; 2814562-75), cytoplasmic extracts were isolated using, the NE-PER® Nuclear and Cytoplasmic Extraction Reagents Kit (Pierce Biotechnology) Probes for biotin pull-down analysis were synthesized as described (Kuwano Y, et al. Mol Cell Biol 2008; 28:45(2-75) using the following PCR primers (sense and antisense, respectively) containing the T7 RNA polymerase promoter sequence CCAAGCTTCTAATACGACTCACTATAGGGAGA (T7) (SEQ ID NO: 1): (T7)GATCTTGCTGAAGACTACAGGC (SEQ ID NO: 2) and TTATTAGCGTCTTTTCAATTCTACAAA (SEQ ID NO: 3) for dCK 3′UTR; (T7)CTCAACGACCACTTTGTCAAGC (SEQ ID NO: 4) and CACAGGGTACTTTATTGATGGTACAT (SEQ ID NO: 5) for GAPD 3′UTR (Casolaro V, et al. J Allergy Clin Immunol 2008; 121:853-9 e4) (see supplemental figure for depiction of UTR positions). Biotinylated probes were synthesized using the MAXIscript T7 kit (Ambion) and Biotinylated dCTP (Enzo Life Sciences). For immunoprecipitation of endogenous RNA-protein complexes (RNP IP) from cytoplasmic (450 μg) extracts, reactions were carried out as described (Kuwano Y, et al. Mol Cell Biol 2008; 28:4562-75), using protein A Sepharose beads (Sigma) that were precoated with 30 μg of either mouse immunoglobulin G1 (IgG1; BD Biosciences), or anti-HuR antibodies. After IP, the RNA in the IP materials was isolated and reverse-transcribed. GAPDH and dCK transcripts were quantified by real-time PCR analysis using each specific primers: AGCAAGGCATTCCTCTTGAA (SEQ ID NO: 6) and CTACAGGCAGCCAAATGGTT (SEQ ID NO: 7) for dCK, TGCACCACCAACTGCTTAGC (SEQ ID NO: 8) and CTCATGACCACAGTCCATGCC (SEQ ID NO: 9) for GAPDH. The relative levels of dCK product was first normalized to GAPDH product in all IP samples, then fold enrichments in HuR IP were compared with IgG IP, as described (Kuwano Y, et al. Mol Cell Biol 2008; 28:4562-75).

Case Selection and Immunohistochemistry

HuR immunostaining was performed on 32 resected PDA specimens from the Thomas Jefferson University pathology archives after IRB approval. All patients received GEM, alone or in combination with XELODA® (Capecitabine) (2 patients), radiation therapy (8 patients) or both (2 patients). The experienced pancreatic pathologist (A.K.W.) reviewed all cases in a blinded fashion and classified the tumors as well differentiated (n=6), moderately differentiated (n=22), or poorly differentiated (n=12). For each case, representative sections were selected for immunohistochemical analysis of HuR cytoplasmic and nuclear staining patterns, which were scored using the following scale: 0 for no staining, 1 for weak and/or focal (<10% of the cells) staining; 2 for moderate or strong, staining (10-50% of the cells); and 3 for moderate or strong staining (>50% of the cells). Combined scores 0 and 1 represented low expression, while combined scores 2 and 3 represented high expression.

HuR Overexpression Preferentially Sensitized Pancreatic Cancer Cell Lines to the Nucleoside Analogs GEM and Ara-C.

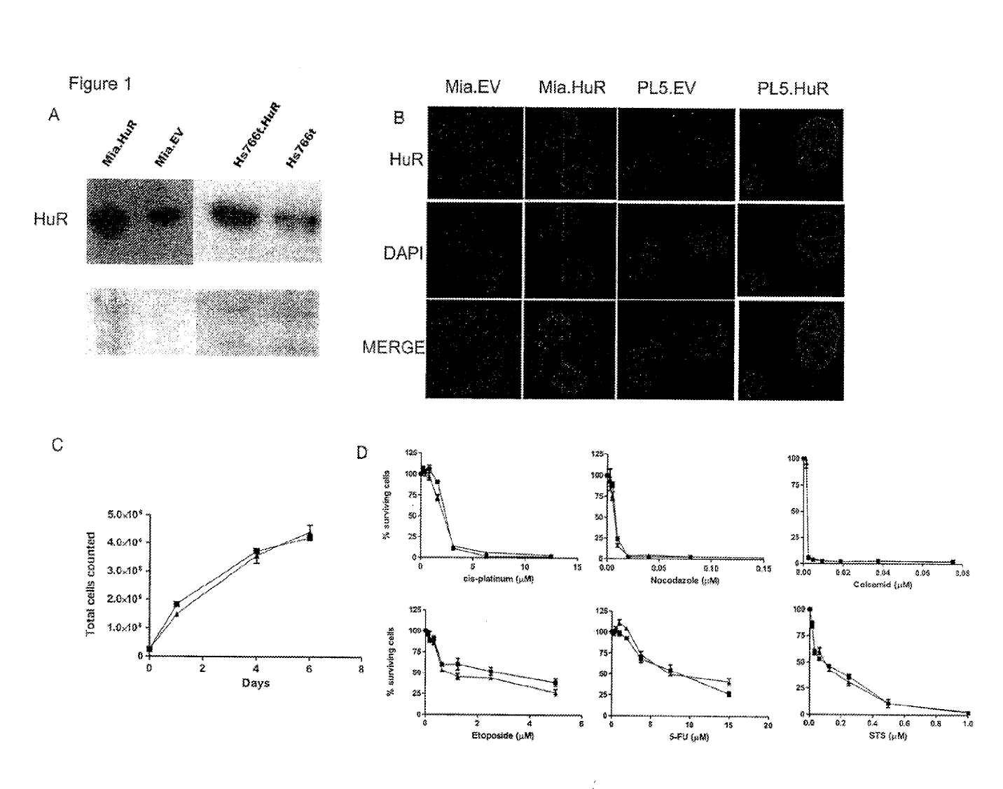

Stable HuR overexpression in the indicated pancreatic cancer cell lines was confirmed by immunoblot and immunofluorescence analyses (FIGS. 1A and B). Contrary to previous studies in colon cancer cell lines (Lopez de Silanes I, et al. Oncogene 2003; 22:7146-54), isogenic, transfected cell lines grew roughly at the same rates (FIG. 1C). Treatment with the indicated chemotherapeutic agents (but not GEM) showed no difference in sensitivity in HuR-overexpressing cells (FIG. 1D).

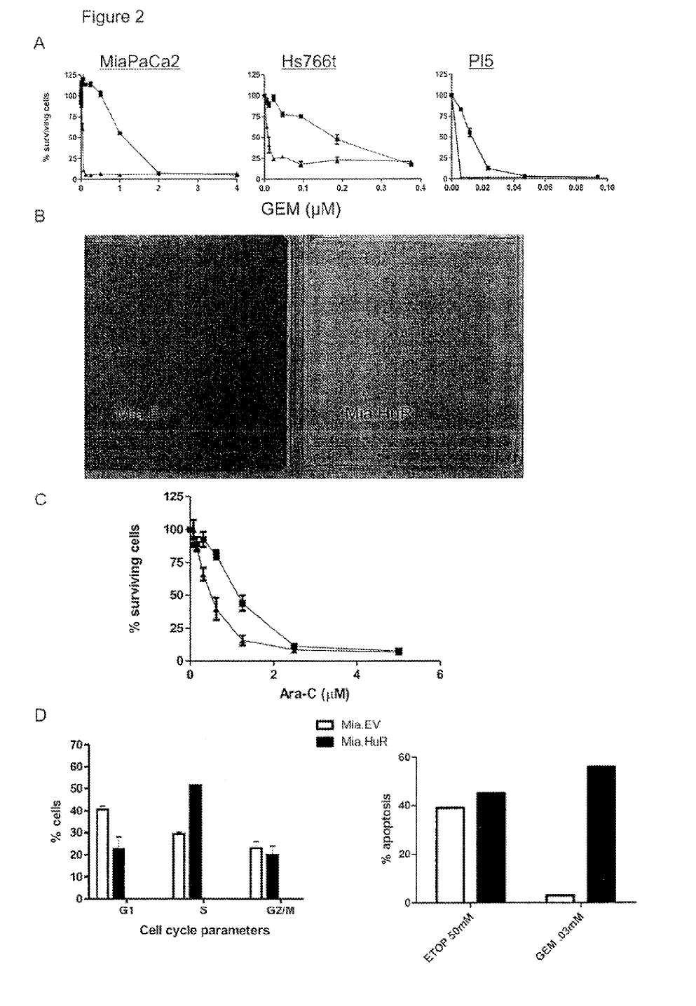

By contrast, cell lines overexpressing HuR were found to be more sensitive to GEM than were control lines, as assessed both by PicoGreen measurement (FIG. 2A) and by staining with crystal violet even when cells were treated with low concentrations of GEM (FIG. 2B). HuR-overexpressing cell lines were similarly selectively more sensitive to Ara-C, another anti-cancer agent that utilizes dCK (FIG. 2C). After GEM treatment, HuR overexpressing cells showed selective enrichment in the S-phase of the cell division cycle and increased apoptosis (FIG. 2D) as compared to the control cells.

HuR Localization and Association with dCK mRNA Upon GEM Treatment.

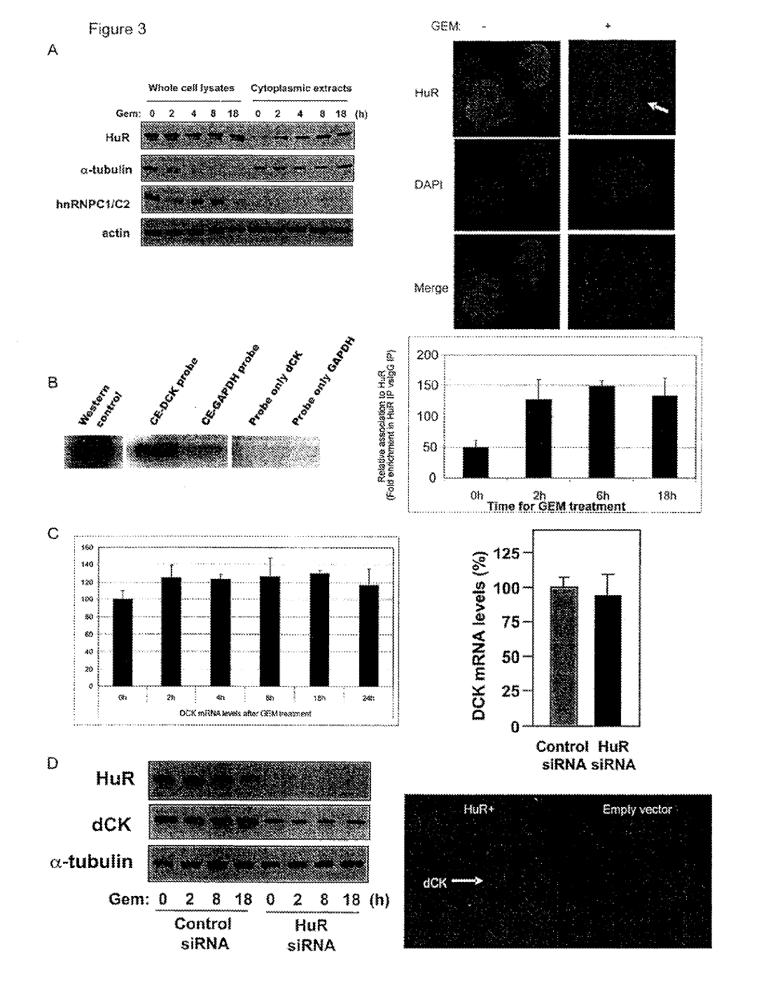

GEM treatment did not alter whole-cell HuR levels in parental MiaPaCa2 cells, but significantly increased the cytoplasmic HuR levels, as determined by immunoblot and immunofluorescence analyses (FIG. 3A). Given that the dCK 3′UTR region contained 8 putative hits of an HuR recognition motif (Lopez de Silanes I, et al. Proc Natl Acad Sci USA 2004; 101:2987-92), the ability of HuR to associate with dCK mRNA was tested using two different RNA-binding assays. First, MiaPaCa2 cytoplasmic extracts were incubated with equimolar amounts of biotinylated transcripts spanning the dCK 3′UTR and the GAPDH 3′UTR (a control RNA, not a target of HuR); the resulting complexes were analyzed by HuR immunoblot. As shown in FIG. 3B (left), HuR bound the dCK 3′UTR much more strongly than the GAPDH 3′UTR. Second, the association of HuR with the endogenous dCK mRNA was tested by using a ribonucleoprotein immunoprecipitation (RNP IP) assay. As shown, while GEM did not alter overall dCK protein or mRNA levels (FIG. 3A,C), it significantly increased HuR's association with dCK mRNA (FIG. 3B, right).

Inhibition of HuR expression using small interfering (si)RNA(7) did not alter dCK miRNA levels (FIG. 3C, right) but decreased dCK protein levels (FIG. 3D, left) regardless of GEM treatment. Conversely, HuR-overexpressing cells displayed higher dCK signals (FIG. 3D, right). Together, these data show that 1) GEM exposure to cancer cells increases cytoplasmic HuR levels (FIG. 3A), 2) HuR associates with dCK mRNA (FIG. 3B), and 3) HuR regulates dCK protein levels (FIG. 3D).

HuR Localization and Expression in PDA Specimens.

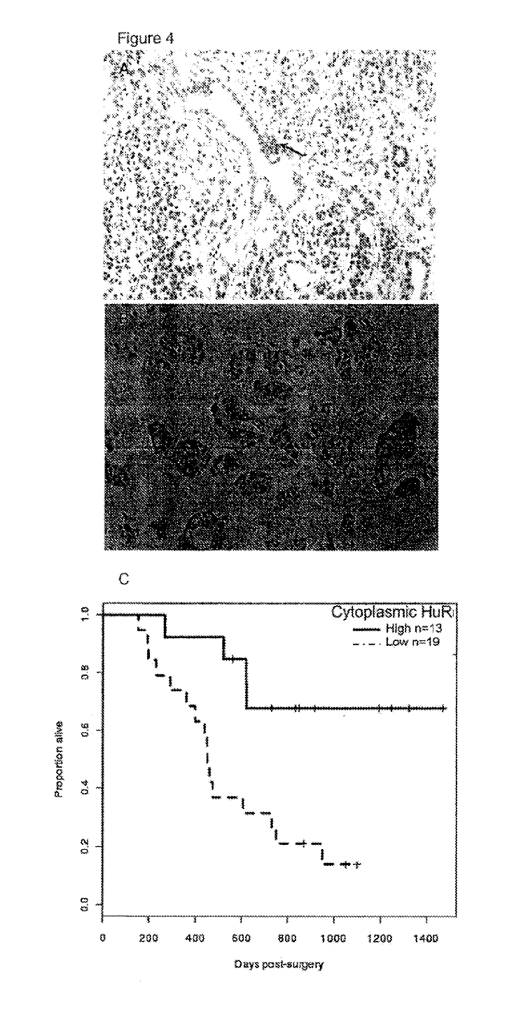

Primarily weak to moderate nuclear HuR expression was detected in normal pancreatic ductal and acinar cells (FIG. 4A). Strong nuclear expression of HuR was found in well-, moderately, and poorly differentiated PDAs. Cytoplasmic HuR accumulation was associated with poorly differentiated pancreatic ductal adenocarcinomas (PDA) (FIG. 4B). FIG. 4C shows the Kaplan-Meier overall survival curves of patients receiving GEM, stratified by their HuR status. The median survival time for patients on GEM was 619 days, with 21 deaths out of the 32 patients who received GEM. A univariate Cox regression model gives a hazard ratio of low to high HuR of 4.48, with a 95% confidence interval of (1.49 to 13.5). Adjusting for age, sex, Xeloda use and radiation therapy in this patient group gives an adjusted hazard ratio of 7.34 (p=0.0022) with a 95% confidence interval of (2.05 to 26.22). These data indicate a greater than 7-fold increase risk of mortality in patients with low cytoplasmic HuR levels (compared to high cytoplasmic HuR levels) among patients receiving GEM, after adjusting for variables as mentioned above.

As elevated cytoplasmic HuR has been correlated with advanced malignancy, the finding that high cytoplasmic HuR levels were associated with an increased therapeutic efficacy of GEM in pancreatic cancer was unexpected. The results that HuR regulates dCK protein concentration and that cytoplasmic HuR levels predict GEM response in the patient cohort indicate that HuR is a key molecule involved in GEM efficacy in cancer. Though not wishing to be bound by any particular theory, HuR's survival repertoire may be to increase dCK levels to process deoxyribonucleosides for survival, however in the presence of nucleoside analogs (such as GEM) HUR's augmentation of dCK is deleterious.

### Example 2

There are a number of potential targets for therapeutic intervention. One molecule targeted antigen showing promise in the treatment of pancreatic cancer is mesothelin (MSLN). This is a particularly good target because it is overexpressed in the majority of pancreatic cancers, but not expressed in the adjacent normal pancreatic tissue surrounding these tumors or in other normal tissues. Over three-quarters of PDA in humans overexpress MSLN. By using the MSLN promoter cancerous cells can effectively be targeted, while sparing normal, healthy tissues.

The approach is to deliver two different genes, each regulated by the MSLN promoter and each having therapeutic potential, to pancreatic tumor cells. One gene is a bioengineered, non-pathogenic diphtheria toxin DNA sequence. For example, in ovarian cancer mouse models (ovarian tumors also overexpress MSLN), nanotherapy is well-tolerated. Delivery of a non-pathogenic diphtheria toxin DNA sequence via nanoparticles significantly reduces tumor burden, and increases the life span of mice when compared to no treatment or conventional chemotherapies. The second gene encodes HuR.

Herein disclosed are in vitro studies that show nanoparticle delivery of a DNA construct that contains the MSLN promoter for cell specific expression of a diphtheria toxin-A (DT-A) gene allows for cancer cell-specific killing.

To reduce off-target expression of DT-A, while maintaining expression in tumor cells, a dual-control regulatory method that targets in a cancer-specific manner can be used. This method makes use of two different tissue-specific promoters, one of which regulates expression of a DNA recombinase. Targeting DNA constructs using the native MSLN and prostate stem cell antigen (PSCA) promoters can be used, both of which are highly active in pancreatic tumor cells relative to normal pancreatic tissue and to other normal tissues. Also a CanScript sequence, an 18 bp enhancer sequence within the native MSLN promoter can be used. Disclosed herein are three copies of the CanScript sequence which can drive gene expression without any other surrounding promoter sequence.

Mesothelin Promoter

MSLN is overexpressed in the majority of pancreatic cancers and has been shown to be overexpressed in a number of other tumor systems, including ovarian cancer. The fact that numerous techniques over a diverse bank of pancreatic tissues have repeatedly shown MSLN overexpression, most likely due to cancer specific transcriptional regulation, provides strong evidence that overexpression of this molecule is a hallmark of pancreatic cancer. Moreover, using a tissue microarray to characterize new MSLN-reactive antibodies, expression of MSLN was not detected in a variety normal tissues including liver, lung, ovarian stroma, brain, breast, and kidney tissues. MSLN expression was observed only in the cancer cells, and in the normal tissue of peritoneal mesothelium and pleural mesothelium. Further, pancreatic cancer precursor lesions express MSLN, thus identifying its expression early in the tumorigenesis process and indicating that it is a therapeutic target.

Transcriptional Targeting and Tight Regulation with the Use of the CanScript and Another Pancreatic Cancer Specific Promoter, Prostate Stem Cell Antigen.

Pancreatic cancer cells can be targeted specifically by utilizing the transcriptional machinery within these cells. The promoter region of MSLN was dissected in order to search for novel molecular events in the process of pancreatic tumorigenesis. Promoter bashing and site-directed mutatagenesis studies of the MSLN promoter revealed a TEF-1 binding site. TEF-1 is part of a family of transcription factors that has multiple functions. A defined sequence, termed CanScript, is responsible for enhanced ‘cancer specific transcription’. A generated construct containing three repeats of this sequence inserted in front of a minimal promoter enhanced cancer-specific transcription nearly 30-fold in a MSLN-expressing pancreatic cancer cell line. Thus, by placing tight transcriptional restrictions on the suicide DNA sequence, transcriptional targeting specifically to pancreatic cancer cells can be ensured. In addition, for added control, a dual-mode of regulating transcription was developed by utilizing a site-directed recombinase. Promoters of two genes that are overexpressed in pancreatic tumor cells, MSLN and the PSCA gene.

An in vivo pancreatic cancer model showed that treatment with a monoclonal anti-PSCA antibody inhibited tumor initiation and growth. Additionally, PSCA has been shown to elicit antibody immune responses in pancreatic cancer patients. Furthermore, a study, attempting to distinguish between ovarian tumors and metastatic pancreatic tumors, found that the majority of metastatic PDAs (n=11) overexpressed both PSCA (82%) and MSLN (72%) proteins.

In Vitro Nanoparticle Delivery of MSLN Promoter-Driven DNA to MSLN+ Pancreatic Cancer Cells.

Using a luciferase reporter gene (Luc) driven by the MSLN promoter, the specificity of the MSLN promoter was accessed in pancreatic cancer cells. MSLN reporter activity was 5.5 times higher in the MSLN+ pancreatic cancer cell line than the MSLN− pancreatic cancer cell line. To adjust for transfection efficiency between the cell lines, these data points were normalized to the relative light unit (RLU) values of cells transfected with CAG/Luc DNA (CAG is a robust, non-specific regulatory sequence consisting of the CMV enhancer and the chicken β-actin promoter).

In Vitro Delivery of DT-A DNA to MSLN+ Pancreatic Cancer Cells Inhibits Protein Translation and Cell Viability.

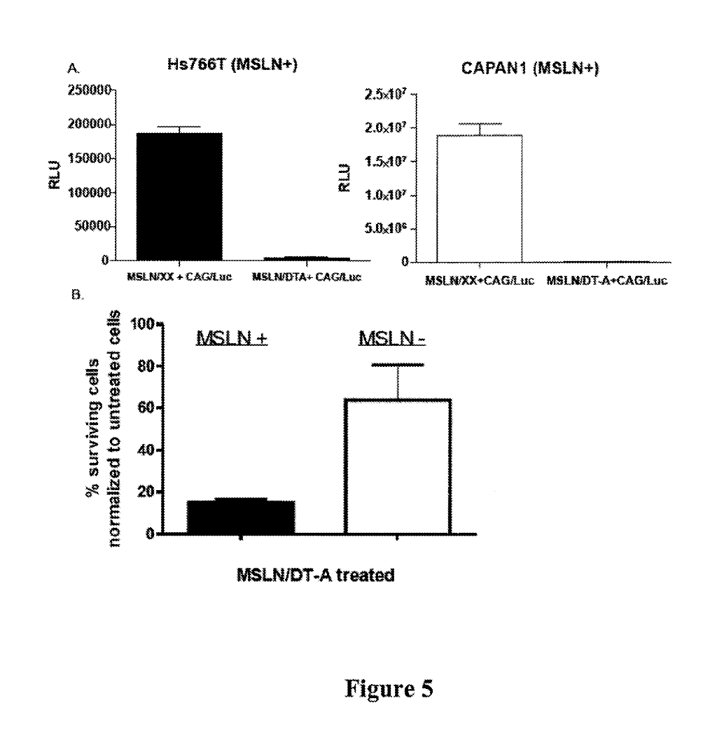

Cells were co-transfected with two DNAs, MSLN/DT-A (MSLN promoter driving DT-A) and CAG/Luc. In these experiments when DT-A expression inhibited translation, luciferase activity is reduced in co-transfected cells. In control transfections, cells were co-transfected with (MSLN/XX+CAG/Luc, XX represents absence of any protein-encoding sequence), or with CAG/Luc alone. Twenty-four hours following co-transfection of the MSLN+ cells with (MSLN/DT-A+CAG/Luc), luciferase activity was reduced >95% as compared to the control, cells co-transfected with (MSLN/XX+CAG/Luc) (FIG. 5A).

An equal number of MSLN+ and MSLN− cells were transfected with MSLN/DT-A nanoparticles and enumerated the viable cells 6 days post-transfection. Control cells were not transfected. MSLN− cells transfected with MSLN/DT-A had a modest reduction in the total number of live cells as compared to cells that received no treatment, while the transfected MSLN+ cell line had nearly an 85% reduction in viable cells as compared to the number of cells in the untreated group (FIG. 5B). Thus, the transfected Hs766T (MSLN+) cells were hypersensitive to the MSLN/DT-A treatment relative to the PL5 (MSLN−) cells (FIG. 5B).

In Vivo Targeted Nanotherapy of MSLN+ Cancer Cells.

C32-MSLN/firefly luciferase DNA (Fluc) nanoparticles were directly injected into subcutaneous xenografts derived from MSLN+ ovarian tumor cells, C32, to poly(β-amino ester) polymer, or PEI was complexed to MSLN/Fluc DNA to generate nanoparticles. Mice were optically imaged and bioluminescence was detected in tumors 6 hrs after injection. In contrast, luciferase expression in tumors injected with PEI-MSLN/Fluc nanoparticles was not detected at 6 hr. post-injection (PEI, polyethylene amine, is a polymer that has been used for many years to deliver DNA. Its use results in significant non-specific cytotoxicity).

Following injection of C32-117-MSLN/Fluc directly into ovarian tumors in a transgenic ovarian mouse tumor model (MISIIR/TAg), whole mice and tumors and individual organs ex vivo were optically imaged, to test for luciferase expression driven by the MSLN promoter in the mice. All injected MSLN+ tumors emitted bioluminescence, indicating that the DNA was successfully delivered to ovarian tumor cells. Bioluminescence was not observed in other organs in the peritoneum.

Intraperitoneal Administration of DT-A Nanoparticles Suppresses Tumor Growth, Increases Life Span of MSLN+ Tumor-Bearing Mice, and does not Target Normal Pancreatic Tissue.

MSLN/DT-A nanoparticles were administered by i.p. injection into MISIIR/TAg mice bearing MSLN+ tumors. Treatment of the ovarian transgenic MISIIR/TAg mice with DT-A nanoparticles increased survival time. 40 mice bearing tumors were identified. Mice were injected twice weekly with either MSLN/DT-A (n=20) or with control MSLN/Fluc nanoparticles (n=20) (in each case, 100 μg DNA/injection). A third control group received no treatment (n=14). The median survival of DT-A-treated mice is significantly longer than either the Fluc-treated mice or untreated mice (78 days vs 64 or 52 days). Of note, some mice in both the DT-A-treated group and the control group withstood nanoparticle treatment for nearly three months. At the termination of the experiment, 30% of mice in the DT-A group were still alive and showed no outward signs of distress. This indicates that i.p. administration of the given dose of DT-A nanoparticles is tolerated quite well by mice. Histological examination of normal tissues including kidney, spleen, lung, stomach, small intestine, large intestine, abdominal wall with peritoneum, diaphragm, urinary bladder, pancreas, uterus and ovary revealed minimal to mild chronic inflammation, no major signs of cellular toxicity, and no pathological changes in normal pancreatic tissues.

A Defined CanScript Sequence, Residing within the MSLN Promoter Increases Cancer Specific Transcription.

Promoter bashing and subsequent site-directed mutagenesis experiments revealed a cancer enhancing transcription sequence, termed a CanScript. Insertion of a plasmid, pGL4-CANx3/luc, containing luciferase and three concatemerized copies of the MSLN CanScript (CANx3) alone resulted in nearly an equal increase in transcription of luciferase under CAG promoter (positive control) regulation in pancreatic cancer cells. This sequence can be utilized to enhance the specificity of expression (presumably by deleting non-specific promoter elements) in MSLN+ cancer cells, thus placing DT-A and HuR expression under tight regulation.

Multiple Pancreatic Cancer Cell Lines are Hypersensitive to Gemcitabine when HuR is Exogenously Introduced. Clinically, HuR Levels are a Biomarker for Gemcitabine Response in Patients.

HuR was stably overexpressed in multiple cell lines (labeled Mia.HuR, see FIG. 1B for characterization). Because HuR has been shown to be activated upon stress, the effect of exogenous HuR expression on drug sensitivity was tested. A number of commonly used chemotherapeutics did not show any difference between the isogenic paired lines (cells overexpressing HuR compared to the control cell line, empty vector alone). However, overexpression of HuR in 3 different pancreatic cancer cell lines renders these cells strikingly hypersensitive to gemcitabine (FIGS. 2A and 2B). Mechanistically, sensitive cells had an S phase cell cycle arrest and underwent apoptosis at IC50 doses. Additionally, HuR bound to the 3′-untranslated region (3′UTR) of the dCK transcript, thus providing mechanistic evidence that overexpression of HuR stabilized the dCK transcript, the enzyme required to convert gemcitabine to an active metabolite in cancer cells. HuR overexpression was stabilized the rate-limiting metabolic step of the prodrug, gemcitabine, and thus increasing the amount of active drug in pancreatic cancer cells. FIG. 4C shows the overall survival curves of patients who received gemcitabine stratified by their HuR status. There is a significant difference in survival between low and high HuR levels (p=0.025). A univariate Cox regression model gives the hazard ratio of low to high HuR of 3.36 with a 95% confidence interval of (1.09, 10.35). Adjusting for age, sex. Xeloda use and radiation therapy in this patient group gave an adjusted hazard ratio of 5.04 (p=0.03) with a 95% confidence interval of (1.15, 22.02). Taken together, this indicated a 5-fold increase in risk of death in patients with low HuR level compared to high HuR levels among patients receiving gemcitabine, adjusting for use of other therapies (FIG. 4C).

Nanoparticle Preparation

Briefly, the polymer is dissolved in DMSO (100 μg/ml). The polymer (1-1.5 mg), is then diluted in 25 μl of 50 mM sodium acetate buffer, pH 5.0, and added to 25 μl DNA suspended in dH20 (2 μg/μl) (1:20 ratio for C32-117), and mixed gently. After incubation of the polymer/DNA mixture at room temperature for 5 min, 10 μl of 30% glucose in PBS is added to the 50 μl polymer/DNA mixture. For administration of nanoparticles to xenografts, 50 μg complexed DNA/60 μl total volume is injected directly into a xenograft tumor using a 30G needle.

Optical Imaging.

A bioluminescence imaging system (IVIS™ Imaging System, Xenogen Corp.) can be used to image mice and detect nanoparticle-delivered Luc gene expression. Using Living Image™ software, the amount of luciferase expression is then quantified and in tumors derived from MSLN+ and MSLN− cells.

TUNEL Assay.

Apoptotic cells can be identified by TUNEL assay using an in Situ Detection Kit (Roche Boehringer Mannheim, Indianapolis, Ind.).

Pancreatic Tumor-Specific Expression of DT-A DNA.

While it was shown herein that the MSLN promoter is very active in MSLN-expressing pancreatic cancer cells, this promoter is leaky and has residual activity in other organs. The ovarian cancer studies show, however, that mice tolerated treatment with MSLN/DT-A nanoparticles very well for extended periods of time, despite off-target DT-A expression. Histopathological studies of multiple organs in these mice show minimal pathology associated with long-term dosing with DT-A nanoparticles. To reduce non-specific expression and possible deleterious side effects as much as possible a dual-control regulatory scheme that can be used to better target expression such as DT-A driven by a prostate-specific promoter. This strategy makes use of two different tissue-specific promoters, one of which regulates expression of a DNA recombinase. The native MSLN and PSCA promoters was used, both of which are highly active in pancreatic tumors and not in normal pancreatic tissue and in other normal tissues. The CanScript sequence, an 18 bp sequence within the native MSLN promoter can also be used. It is shown that three concatemerized copies of the CanScript can induce gene transcription in pancreatic cancer cells). Using only this sequence removes unwanted enhancer elements active in normal cells, thus increasing cancer cell specificity.

Generation of DNA Constructs for Optimized Targeting.

The CanScript (CANx3) alone can drive gene expression such as in the case of, for example, a CANX3-DTA or CANX3-HuR construct. Thus, the efficacy of the Canx3 alone driving DT-A expression in MSLN+ and MSLN− pancreatic cancer cells can be tested. Either the intact native MSLN promoter sequence or CANX3 can be used to regulate the expression of Cre recombinase (Cre). For example, the construct can comprise PSCA promoter-LoxP-Cre-CANX3-LoxP-DT-A, PSCA promoter-LoxP-Cre-MSLN-LoxP-DT-A, PSCA promoter-LoxP-Cre-CANX3-LoxP-HuR, or PSCA promoter-LoxP-Cre-MSLN-LoxP-HuR. When Cre is expressed in cells, the Cre-encoding DNA is excised from the construct and, and as a consequence of this DNA recombination, the second pancreatic tumor-specific promoter, PSCA, is juxtaposed to the DT-A or HuR sequence, thereby allowing for DT-A or HuR expression. Hence, transcriptional regulation is combined with Cre recombinase-mediated DNA recombination to safeguard against expression of DT-A in non-cancerous tissue. The 18 bp CanScript concatomer sequence (CANx3) has been successfully sub-cloned into expression constructs, along with the PSCA promoter (the sequence was kindly donated by Dr. Robert Reiter, UCLA). Comparable constructs containing the luciferase (Luc) sequence in place of DT-A allows the use of optical imaging to evaluate gene expression in multiple organs easily.

### 5.3. REFERENCES

Abdelmohsen K, Lal A, Kim H H, Gorospe M. Posttranscriptional orchestration of an anti-apoptotic program by HuR. Cell Cycle 2007; 6:1288-92.
Anderson D G, Peng W, Akinc A, et al. A polymer library approach to suicide gene therapy for cancer. Proc Natl Acad Sci USA 2004; 101:16028-33.
Antic D, Keene J D. Embryonic lethal abnormal visual RNA-binding proteins involved in growth, differentiation, and posttranscriptional gene expression. Am J Hum Genet 1997; 61:273-8.
Antani P, Iacobuzio-Donahue C, Ryu B, et al. Mesothelin is overexpressed in the vast majority of ductal adenocarcinomas of the pancreas: identification of a new pancreatic cancer marker by serial analysis of gene expression (SAGE). Clin Cancer Res 2001; 7:3862-8.
Argani P, Rosty C, Reiter R E, et al. Discovery of new markers of cancer through serial analysis of gene expression: prostate stem cell antigen is overexpressed in pancreatic adenocarcinoma. Cancer Res 2001, 61:4320-4.
Arteaga, C., Targeting HER1/EGFR: a molecular approach to cancer therapy. Semin Oncol, 2003. 30(3 Suppl 7): p. 3-14.
Bai, J., N. Sata, and H. Nagai, Gene expression analysis for predicting gemcitabine sensitivity in pancreatic cancer patients. HPB (Oxford), 2007. 9(2): p. 150-5.
Bhowmick, N. A., E. G. Neilson, and H. L. Moses, Stromal fibroblasts in cancer initiation and progression. Nature, 2004. 432(7015): p. 332-7.
Bilimoria K Y, Bentrem D J, Ko C Y, Stewart A K, Winchester D P, Talamonti M S. National failure to operate on early stage pancreatic cancer. Ann Surg 2007; 246:173-80.
Bissell, M. J. and D. Radisky, Putting tumours in context. Nat Rev Cancer, 2001. 1(1): p. 46-54.
Boussif O, Lezoualc'h F, Zama M A, et al. A versatile vector for gene and oligonucleotide transfer into cells in culture and in vivo: polyethylenimine. Proc Natl Acad Sci USA 1995; 92:7297-301.
Brody J R, Witkiewicz A, Williams T K, et al. Reduction of pp32 expression in poorly differentiated pancreatic ductal adenocarcinomas and intraductal papillary mucinous neoplasms with moderate dysplasia, Mod Pathol 2007; 20:1238-44.
Burris H A, 3rd, Moore M J, Andersen J, et al. Improvements in survival and clinical benefit with gemcitabine as first-line therapy for patients with advanced pancreas cancer: a randomized trial. J Clin Oncol 1997; 15:2403-13.
Cao D, Ji H, Ronnett B M. Expression of mesothelin, fascin, and prostate stem cell antigen in primary ovarian mucinous tumors and their utility in differentiating primary ovarian mucinous tumors from metastatic pancreatic mucinous carcinomas in the ovary. Int J Gynecol Pathol 2005; 24:67-72.
Casolaro V, Fang X, Tancowny B, et al. Posttranscriptional regulation of IL-13 in T cells: role of the RNA-binding protein HuR. J Allergy Clin Immunol 2008; 121:853-9 e4.
Chang K, Pastan I. Molecular cloning and expression of a cDNA encoding a protein detected by the K1 antibody from an ovarian carcinoma (OVCAR-3) cell line. Int J Cancer 1994; 57:90-7.
Collier R J. Diphtheria toxin: mode of action and structure. Bacteriol Rev 1975; 39:54-85.
Connolly D C, Bao R, Nikitin A Y, et al. Female mice chimeric for expression of the simian virus 40 TAg under control of MISIIR promoter develop epithelial ovarian cancer. Cancer Res 2003; 63:1389-97.
Denkert C, Weichert W, Pest S, et al. Overexpression of the embryonic-lethal abnormal vision-like protein HuR in ovarian carcinoma is a prognostic factor and is associated with increased cyclooxygenase 2 expression. Cancer Res 2004; 64:189-95.
Denkert C, Weichert W. Winzer K J, et al. Expression of the ELAV-like protein HuR is associated with higher tumor grade and increased cyclooxygenase-2 expression in human breast carcinoma. Clin Cancer Res 2004; 10:5580-6.
Engelman, J. A., et al., Recombinant expression of caveolin-1 in oncogenically transformed cells abrogates anchorage-independent growth. J Biol Chem, 1997. 272(26): p. 16374-81.
Frantz, S., Drug discovery: playing dirty. Nature, 2005. 437(7061): p. 942-3.
Frierson H F, Jr., Moskaluk C A, Powell S M, et al. Large-scale molecular and tissue microarray analysis of mesothelin expression in common human carcinomas. Hum Pathol 2003; 34:605-9.
Galbiati, F., et al., Targeted downregulation of caveolin-1 is sufficient to drive cell transformation and hyperactivate the p42/44 MAP kinase cascade. EMBO J, 1998. 17(22): p. 6633-48.
Gallmeier E, Calhoun E S, Rago C, et al. Targeted disruption of FANCC and FANCG in human cancer provides a preclinical model for specific therapeutic options. Gastroenterology 2006; 130:2145-54.
Giles, K. M., et al., The 3′-untranslated region of p21WAF1 mRNA is a composite cis-acting sequence bound by RNA-binding proteins from breast cancer cells, including HuR and poly(C)-binding protein. J Biol Chem, 2003. 278(5): p. 2937-46.
Gioyannetti, E., et al, Transcription analysis of human equilibrative nucleoside transporter-1 predicts survival in pancreas cancer patients treated with gemcitabine. Cancer Res, 2006. 66(7): p. 3928-35.
Good P J. A conserved family of elav-like genes in vertebrates. Proc Natl Acad Sci USA 1995; 92:4557-61.
Gregoire, V., et al., Role of deoxycytidine kinase (dCK) activity in gemcitabine's radioenhancement in mice and human cell lines in vitro. Radiother Oncol. 2002. 63(3): p. 329-38.
Grubbs E G, Abdel-Wahab Z, Tyler D S, Pruitt S K. Utilizing quantitative polymerase chain reaction to evaluate prostate stem cell antigen as a tumor marker in pancreatic cancer. Ann Surg Oncol 2006; 13:1645-54.
Gu Z, Yamashiro J, Kono E, Reiter R E. Anti-prostate stem cell antigen monoclonal antibody 1G8 induces cell death in vitro and inhibits tumor growth in vivo via a Fc-independent mechanism. Cancer Res 2005; 65:9495-500.
Hassan R, Laszik Z G, Lerner M, Raffeld M, Postier R, Brackett D. Mesothelin is overexpressed in pancreaticobiliary adenocarcinomas but not in normal pancreas and chronic pancreatitis. Am J Clin Pathol 2005; 124:838-45.
Hassan R, Viner J L, Wang Q C, Margulies I, Kreitman R J, Pastan I. Anti-tumor activity of K1-LysPE38QQR, an immunotoxin targeting mesothelin, a cell-surface antigen overexpressed in ovarian cancer and malignant mesothelioma. J Immunother 2000; 23:473-9.
Heinonen M, Fagerholm R. Aaltonen K, et al, Prognostic role of HuR in hereditary breast cancer. Clin Cancer Res 2007; 13:6959-63.
Herrmann, R., et al., Gemcitabine plus capecitabine compared with gemcitabine alone in advanced pancreatic cancer: a randomized, multicenter, phase III trial of the Swiss Group for Clinical Cancer Research and the Central European Cooperative Oncology Group. J Clin Oncol, 2007. 25(46): p. 2212-7.
Hertel L W, Boder G B, Kroin J S, et al. Evaluation of the antitumor activity of gemcitabine (2′,2′-difluoro-2′-deoxycytidine). Cancer Res 1990; 50:4417-22.
Hingorani S R, Wang L, Multani A S, et al. Trp53R172H and KrasG12D cooperate to promote chromosomal instability and widely metastatic pancreatic ductal adenocarcinoma in mice. Cancer Cell 2005; 7:469-83.
Hinman M N, Lou H. Diverse molecular functions of Hu proteins. Cell Mol Life Sci 2008; 65:3168-81.
Hnasko, R. and M. P. Lisanti, The biology of caveolae: lessons from caveolin knockout mice and implications for human disease. Mol Interv, 2003. 3(8): p. 445-64.
Hostetter C, Licata L A, Witkiewicz A, et al. Cytoplasmic accumulation of the RNA binding protein HuR is central to tamoxifen resistance in estrogen receptor positive breast cancer cells. Cancer Biol Ther 2008; 7:1496-506.
Hostetter, C., et al., Cytoplasmic accumulation of the RNA binding protein HuR is central to tamoxifen resistance in estrogen receptor positive breast cancer cells. Cancer Biol Ther, 2008. 7(9): p. 1496-506.
Hruban R H, Iacobuzio-Donahue C, Wilentz R E, Goggins M, Kern S E. Molecular pathology of pancreatic cancer. Cancer J 2001; 7:251-8.
Hruban R H, Rustgi A K, Brentnall T A, Tempero M A, Wright C V, Tuveson D A. Pancreatic cancer in mice and man: the Penn Workshop 2004. Cancer Res 2006; 66: 14-7.
Hucl T, Brody J R, Gallmeier E, Iacobuzio-Donahue C A, Farrance I K, Kern S E. High cancer-specific expression of mesothelin (MSLN) is attributable to an upstream enhancer containing a transcription enhancer factor dependent MCAT motif. Cancer Res 2007; 67:9055-65.
Hucl T, Gallmeier E, Kern S E. Distinguishing rational from irrational applications of pharmacogenetic synergies from the bench to clinical trials. Cell Cycle 2007; 6:1336-41.
Iacobuzio-Donahue C A, Ashfaq R, Maitra A, et al. Highly expressed genes in pancreatic ductal adenocarcinomas: a comprehensive characterization and comparison of the transcription profiles obtained from three major technologies. Cancer Res 2003; 63:8614-22.
Itakura, J., et al., Enhanced expression of vascular endothelial growth factor in human pancreatic cancer correlates with local disease progression, Clin Cancer Res, 1997. 3(8): p. 1309-16.
Jemal, A., et al., Cancer statistics, 2007. CA Cancer J Clin, 2007. 57(1): p. 43-66.
Jimeno, A., et al., Coordinated epidermal growth factor receptor pathway gene overexpression predicts epidermal growth factor receptor inhibitor sensitivity in pancreatic cancer. Cancer Res, 2008. 68(8): p. 2841-9.
Jones, S., et al., Core signaling pathways in human pancreatic cancers revealed by global genomic analyses. Science, 2008. 321(5897): p. 1801-6.
Kalluri, R. and M. Zeisberg, Fibroblasts in cancer. Nat Rev Cancer, 2006, 6(5): p. 392-401.
Kim, J. B., R. Stein, and M. J. O'Hare, Tumour-stromal interactions in breast cancer: the role of stroma in tumorigenesis. Tumour Biol, 2005, 26(4): p. 173-85.
Kindler, H. L., et al., Phase II trial of bevacizumab plus gemcitabine in patients with advanced pancreatic cancer. J Clin Oncol, 2005. 23(31): p. 8033-40.
King P H, Levine T D, Fremeau R T, Jr., Keene J D. Mammalian homologs of Drosophila ELAV localized to a neuronal subset can hind in vitro to the 3′ UTR of mRNA encoding the Id transcriptional repressor. J Neurosci 1994; 14:1943-52.
Kleeff J, Michalski C W, Friess H, Buchler M W. Surgical treatment of pancreatic cancer: the role of adjuvant and multimodal therapies, Eur I Surg Oncol 2007; 33:817-23.
Koleske, A. J., D. Baltimore, and M. P. Lisanti, Reduction of caveolin and caveolae in oncogenically transformed cells, Proc Natl Acad Sci USA, 1995. 92(5): p. 1381-5.
Kullmann, M., et al., ELAV/Hu proteins inhibit p27 translation via an IRES element in the p27 5′UTR. Genes Dev, 2002. 16(23): p. 3087-99.
Kuwano Y. Kim H H, Abdelmohsen K, et at MKP-1 mRNA stabilization and translational control by RNA-binding proteins HuR and NF90. Mol Cell Biol 2008; 28:4562-75.
LeBaron, M. J., et al., In vivo response-based identification of direct hormone target cell populations using high-density tissue arrays. Endocrinology, 2007. 148(3): p. 989-1008.
LeBaron, M. J., et al., Ultrahigh density microarrays of solid samples Nat Methods, 2005. 2(7): p. 511-3.
Li M, Bharadwaj U, Zhang R, et al. Mesothelin is a malignant factor and therapeutic vaccine target for pancreatic cancer. Mol Cancer Ther 2008; 7:286-96.
Li Z R, Campbell J, Rustum Y M. Effect of 3-deazauridine on the metabolism, toxicity, and antitumor activity of azacitidine in mice bearing L1210 leukemia sensitive and resistant to cytarabine. Cancer Treat Rep 1983; 67:547-54.
Li, Z., et al., Alternate cyclin D1 mRNA splicing modulates p27KIP1 binding and cell migration. J Biol Chem, 2008. 283(11): p. 7007-15.
Lopez de Silanes I, Fan J, Galban C J, Spencer R G, Becker K G, Gorospe M. Global analysis of HuR-regulated gene expression in colon cancer systems of reducing complexity. Gene Expr 2004; 12:49-59.
Lopez de Silanes I, Fan J, Yang X, et al. Role of the RNA-binding protein HuR in colon carcinogenesis. Oncogene 2003; 22:7146-54.
Lopez de Silanes I, Lal A, Gorospe M. HuR: post-transcriptional paths to malignancy. RNA Biol 2005; 2:11-3.
Lopez de Silanes I, Zhan M, Lal A, Yang X, Gorospe M. Identification of a target RNA motif for RNA-binding protein HuR Proc Natl Acad Sci USA 2004; 101:2987-92.
Luo D, Saltzman W M. Synthetic DNA delivery systems. Nat Biotechnol 2000; 18:33-7.
Ma W J, Cheng S, Campbell C, Wright A, Furneaux H. Cloning and characterization of HuR, a ubiquitously expressed Elav-like protein. J Biol Chem 1996; 271:8144-51.
Maitra A, Adsay N V, Argani P, et al. Multicomponent analysis of the pancreatic adenocarcinoma progression model using a pancreatic intraepithelial neoplasia tissue microarray. Mod Pathol 2003; 16:902-12.
Maxwell I H, Maxwell F, Glode L M. Regulated expression of a diphtheria toxin A-chain gene transfected into human cells: possible strategy for inducing cancer cell suicide. Cancer Res 1986; 46:4660-4.
Mazan-Mamczarz K, Galban S, Lopez de Silanes I, et al. RNA-binding protein HuR enhances p53 translation in response to ultraviolet light irradiation. Proc Natl Acad Sci USA 2003; 100:8354-9.
Mazroui R, Di Marco S, Clair E, et al. Caspase-mediated cleavage of HuR in the cytoplasm contributes to pp32/PHAP-I regulation of apoptosis. J Cell Biol 2008; 180:113-27.
Mercier, I., et al., Human breast cancer-associated fibroblasts (CAFs) show caveolin-1 downregulation and RB tumor suppressor functional inactivation: Implications for the response to hormonal therapy. Cancer Biol Ther, 2008. 7(8): p. 1212-25.
Millard S S, Vidal A, Markus M, Koff A. A U-rich element in the 5′ untranslated region is necessary for the translation of p27 mRNA. Mol Cell Biol 2000; 20:5947-59.
Miller, V. A., et al., Bronchioloalveolar pathologic subtype and smoking history predict sensitivity to gefitinib in advanced non-small-cell lung cancer. J Clin Oncol, 2004. 22(6): p. 1103-9.
Moore, M. J., et al. Erlotinib plus gemcitabine compared with gemcitabine alone in patients with advanced pancreatic cancer: a phase III trial of the National Cancer Institute of Canada Clinical Trials Group. J Clin Oncol, 2007. 25(15): p. 1960-6.
Mueller, M. M. and N. E. Fusenig, Friends or foes—bipolar effects of the tumour stroma in cancer. Nat Rev Cancer, 2004. 4(11): p. 839-49.
Nevalainen, M. T., et al., Signal transducer and activator of transcription-5 activation and breast cancer prognosis. J Clin Oncol, 2004. 22(11): p. 2053-60.
Noble S, Goa K L. Gemcitabine. A review of its pharmacology and clinical potential in non-small cell lung cancer and pancreatic cancer. Drugs 1997; 54:447-72.
Oettle, H., Post, S., Neuhaus, P., Gellert, K., Langrehr, J., et al. (2007) Jama 297, 267-277
Olive K P, Tuveson D A. The use of targeted mouse models for preclinical testing of novel cancer therapeutics. Clin Cancer Res 2006; 12:5277-87.
Olumi, A. F., et al., Carcinoma-associated fibroblasts direct tumor progression of initiated human prostatic epithelium. Cancer Res, 1999. 59(19): p. 5002-11.
Onda M, Willingham M, Nagata S, et al. New monoclonal antibodies to mesothelin useful for immunohistochemistry, fluorescence-activated cell sorting, Western blotting, and ELISA. Clin Cancer Res 2005; 11:5840-6.
Peng W, Anderson D G, Bao Y, Padera R F, Jr., Langer R, Sawicki J A. Nanoparticulate delivery of suicide DNA to murine prostate and prostate tumors. Prostate 2007; 67:855-62.
Peng W, Chen J, Huang Y H, Sawicki J A. Tightly-regulated suicide gene expression kills PSA-expressing prostate tumor cells. Gene Ther 2005; 12: 1573-80.
Peng W, Verbitsky A, Bao Y, Sawicki J. Regulated expression of diphtheria toxin in prostate cancer cells. Mol Ther 2002; 6:537-45.
Razani, B., et al., Caveolin-1 null mice are viable but show evidence of hyperproliferative and vascular abnormalities. J Biol Chem, 2001. 276(41): p. 38121-38.
Rimm, D. L., Tissue microarrays without cores. Nat Methods, 2005. 2(7): p. 492-3.
Rodriguez J A, Li M, Yao Q, Chen C, Fisher W E. Gene overexpression in pancreatic adenocarcinoma: diagnostic and therapeutic implications. World J Surg 2005; 29:297-305.
Rosen D G, Wang L, Atkinson J N, et al Potential markers that complement expression of CA125 in epithelial ovarian cancer. Gynecol Oncol 2005; 99:267-77.
Rubio-Viqueira B, Jimeno A, Cusatis G, et al. An in vivo platform for translational drug development in pancreatic cancer. Clin Cancer Res 2006; 12:4652-61.
Rui, H. and M. J. Lebaron, Creating tissue microarrays by cutting-edge matrix assembly. Expert Rev Med Devices, 2005. 2(6): p. 673-80.
Ryu B, Jones J, Blades N J, et al. Relationships and differentially expressed genes among pancreatic cancers examined by large-scale serial analysis of gene expression. Cancer Res 2002; 62:819-26.
Sato N, Fukushima N, Maitra A, et al, Discovery of novel targets for aberrant methylation in pancreatic carcinoma using high-throughput microarrays. Cancer Res 2003; 63:3735-42.
Sato N, Fukushima N, Maitra A, et al. Gene expression profiling identifies genes associated with invasive intraductal papillary mucinous neoplasms of the pancreas. Am J Pathol 2004; 164:903-14.
Sawicki J A, Monks B, Morris R J. Cell-specific ecdysone-inducible expression of FLP recombinase in mammalian cells. Biotechniques 1998, 25:868-70, 72-5.
Scholler N, Fu N, Yang Y, et al. Soluble member(s) of the mesothelin/megakaryocyte potentiating factor family are detectable in sera from patients with ovarian carcinoma. Proc Natl Acad Sci USA 1999; 96:11531-6.
Sebastiani V, Ricci F, Rubio-Viqueira B, et al. Immunohistochemical and genetic evaluation of deoxycytidine kinase in pancreatic cancer: relationship to molecular mechanisms of gemcitabine resistance and survival. Clin Cancer Res 2006; 12:2492-7.
Sener S F, Fremgen A, Menck H R, Winchester D P. Pancreatic cancer: a report of treatment and survival trends for 100,313 patients diagnosed from 1985-1995, using the National Cancer Database. J Am Coll Surg 1999; 189:1-7.
Sengupta, S., et al., The RNA-binding protein HuR regulates the expression of cyclooxygenase-2. J Biol Chem, 2003. 278(27): p. 25227-33.
Seo, Y., et al., High expression of vascular endothelial growth factor is associated with liver metastasis and a poor prognosis for patients with ductal pancreatic adenocarcinoma. Cancer, 2000. 88(10): p. 2239-45.
Serini, G. and G. Gabbiani, Mechanisms of myofibroblast activity and phenotypic modulation. Exp Cell Res, 1999. 250(2): p. 273-83.
Showalter, S. L., et al., Nanoparticulate delivery of diphtheria toxin DNA effectively kills Mesothelin expressing pancreatic cancer cells. Cancer Biol Ther, 2008. 7(10): p. 1584-90.
S L Showalter Y-H H, A Witkiewicz, C L Costantino, C L Costantino, C J Yeo, J J Green, R Langer, D G Anderson, J A Sawicki and J R Brody. Nanoparticulate delivery of diphtheria toxin DNA effectively kills mesothelin expressing pancreatic cancer cells. Cancer Biology and Therapy 2008; 7.
Somia N, Verma I M. Gene therapy: trials and tribulations. Nat Rev Genet 2000; 1:91-9.
Sotgia, F., et al., Caveolin-1, mammary stem cells, and estrogen-dependent breast cancers. Cancer Res, 2006, 66(22): p. 10647-51.
Szabo A, Dalmau J, Manley G, et al. HuD, a paraneoplastic encephalomyelitis antigen, contains RNA-binding domains and is homologous to Elav and Sex-lethal. Cell 1991; 67:325-33.
Tanaka M, Komatsu N, Terakawa N, et al. Increased levels of IgG antibodies against peptides of the prostate stem cell antigen in the plasma of pancreatic cancer patients. Oncol Rep 2007; 18:161-6.
Tobita, K., et al., Epidermal growth factor receptor expression in human pancreatic cancer: Significance for liver metastasis, Int J Mol Med, 2003. 11(3): p. 305-9.
Trusheim, M. R., E. R. Berndt, and F. L. Douglas, Stratified medicine: strategic and economic implications of combining drugs and clinical biomarkers. Nat Rev Drug Discov, 2007. 6(4): p. 287-93.
van der Heijden M S, Brody J R, Dezentje D A, et al. In vivo therapeutic responses contingent on Fanconi anemia/BRCA2 status of the tumor. Clin Cancer Res 2005; 11:7508-15.
Wang W, Furneaux H, Cheng H, et al. HuR regulates p21 mRNA stabilization by UV light. Mol Cell Biol 2000; 20:760-9.
Warshaw, A. L. and C. Fernandez-del Castillo, Pancreatic carcinoma N Engl J Med, 1992. 326(7): p. 455-65.
Watanabe H, Okada G, Ohtsubo K, et al. Expression of mesothelin mRNA in pure pancreatic juice from patients with pancreatic carcinoma, intraductal papillary mucinous neoplasm of the pancreas, and chronic pancreatitis. Pancreas 2005; 30:349-54.
Wente M N, Jain A, Kono E, et al. Prostate stem cell antigen is a putative target for immunotherapy in pancreatic cancer. Pancreas 2005; 31:119-25.
Williams, T. M. and M. P. Lisanti, Caveolin-1 in oncogenic transformation, cancer, and metastasis, Am J Physiol Cell Physiol, 2005. 288(3): p. C494-506.
Williams, T. M., et al., Stromal and epithelial caveolin-1 both confer a protective effect against mammary hyperplasia and tumorigenesis: Caveolin-1 antagonizes cyclin D1 function in mammary epithelial cells. Am J Pathol, 2006. 169(5): p. 1784-801.
Witkiewicz, A., et al., Expression of indoleamine 2,3-dioxygenase in metastatic pancreatic ductal adenocarcinoma recruits regulatory T cells to avoid immune detection. J Am Coll Surg, 2008. 206(5): p. 849-54; discussion 854-6.
Witkiewicz. A. K., et al, Co-expression of fatty acid synthase and caveolin-1 in pancreatic ductal adenocarcinoma: implications for tumor progression and clinical outcome. Cell Cycle, 2008. 7(19): p. 3021-5.
Yang, S. X., et al, Gene expression profile and angiogenic marker correlates with response to neoadjuvant bevacizumab followed by bevacizumab plus chemotherapy in breast cancer. Clin Cancer Res, 2008. 14(18): p. 5893-9.
Yoo P S, Sullivan C A, Kiang S, et al. Tissue Microarray Analysis of 560 Patients with Colorectal Adenocarcinoma: High Expression of HuR Predicts Poor Survival, Ann Surg Oncol 2008.

1-44. (canceled)

45. A method of assessing the efficacy of a nucleoside analog treatment of cancer in a subject comprising measuring the expression level and/or activity level of Human Antigen R (HuR) in a biological sample obtained from said subject,
wherein an elevated level of HuR expression and/or activity in the cells of the biological sample relative to normal cells or a non-responding subject indicates that the subject is responsive to said nucleoside analog treatment.

46. The method of claim 45, wherein said nucleoside analog is selected from the group consisting of gemcitabine (GEM), cytarabine (Ara-C), clofarabine, BCH-4556, troxacitabine, vidarabine, zidovudine, and 1-(2-deoxy-2-fluoro-4-thio-β-D-arabinofuranosyl)cytosine (4′-thio-FAC).

47. The method of claim 45, wherein said nucleoside analog is gemcitabine.

48. The method of claim 45, wherein said nucleoside analog is cytarabine (Ara-C).

49. The method of claim 45, wherein the HuR is cytoplasmic HuR.

50. The method of claim 45, wherein an elevated expression level of HuR correlates to the subject being responsive to said nucleoside analog treatment.

51. The method of claim 45, wherein an elevated activity level of HuR correlates to the subject being responsive to said nucleoside analog treatment.

52. The method of claim 45, wherein a reduced expression or activity level of HuR relative to normal cells or anon-responding subject is correlated with the subject being resistant to said nucleoside analog treatment.

53. The method of claim 45, wherein said biological sample is a tumor sample.

54. The method of claim 45, wherein said biological sample is from a biopsy or surgical resection.

55. The method of claim 45, wherein the level of expression and/or activity of HuR is measured by immunohistochemistry, immunoprecipitation, or real time PCR.

56. The method of claim 45, wherein the cancer is selected from the group consisting of pancreatic cancer, small cell lung cancer, colorectal, head and neck cancer, ovarian cancer, melanoma, renal cell carcinoma, non-small cell lung cancer, bladder cancer, ooesophageal cancer, leukemia, lymphoma, and gastric cancer.

57. The method of claim 45, wherein an elevated level of cytoplasmic HuR expression compared to negative cytoplasmic HuR expression levels is correlated with an increased therapeutic efficacy of the nucleoside analog treatment.

58. The method of claim 56, wherein the subject has pancreatic cancer.

59. A method of enhancing the efficacy of a nucleoside analog treatment of a cancer subject comprising increasing the expression or activity level of HuR in said subject.

60. The method of claim 59, wherein the HuR is cytoplasmic HuR.

61. The method of claim 59, wherein the subject is administered the nucleoside analog and HuR.

62. The method of claim 59, wherein the subject is co-administered the nucleoside analog and a polynucleotide construct encoding for HuR.

63. The method of claim 59, wherein the subject is first administered a polynucleotide construct encoding for HuR and then administered the nucleoside analog.

64. The method of claim 59, wherein the subject is first administered the nucleoside analog and then administered a polynucleotide construct encoding for HuR.

65. The method of claim 59, wherein the subject has pancreatic cancer, small cell lung cancer, colorectal, head and neck cancer, ovarian cancer, melanoma, renal cell carcinoma, non-small cell lung cancer, bladder cancer, oesophageal cancer, lymphoma, leukemia, or gastric cancer.

66. The method of claim 65, wherein the subject has pancreatic cancer.

67. The method of claim 59, wherein said nucleoside analog is selected from the group consisting of gemcitabine (GEM), cytarabine (Ara-C), clofarabine, BCH-4556, troxacitabine, vidarabine, zidovudine, and 1-(2-deoxy-2-fluoro-4-thio-β-D-arabinofuranosyl)cytosine (4′-thio-FAC).

68. The method of claim 59, wherein said nucleoside analog is gemcitabine.

69. The method of claim 59, wherein said nucleoside analog is cytarabine (Ara-C).

70. A composition comprising a nucleoside analog and a polynucleotide construct encoding for HuR.

71. The composition of claim 70, wherein the construct comprises SEQ ID NO: 11.

72. The composition of claim 70, wherein the polynucleotide construct further comprises the MSLN promoter.

73. The composition of claim 70, wherein said nucleoside analog is selected from the group consisting of gemcitabine (GEM), cytarabine (Ara-C), clofarabine, BCH-4556, troxacitabine, vidarabine, zidovudine, and 1-(2-deoxy-2-fluoro-4-thio-β-D-arabinofuranosyl)cytosine (4′-thio-FAC).

74. The composition of claim 70, wherein said nucleoside analog is gemcitabine.

75. The composition of claim 70, wherein said nucleoside analog is cytarabine (Ara-C).


SEQUENCE LISTING (14 sequences)

SEQ ID NO: 1 - DNA, 32 bp, Artificial Sequence
  PCR primer
ccaagcttctaatacgactcactatagggaga

SEQ ID NO: 2 - DNA, 22 bp, Artificial Sequence
  PCR primer
gatcttgctgaagactacaggc

SEQ ID NO: 3 - DNA, 27 bp, Artificial Sequence
  PCR primer
ttattagcgtcttttcaattctacaaa

SEQ ID NO: 4 - DNA, 22 bp, Artificial Sequence
  PCR primer
ctcaacgaccactttgtcaagc

SEQ ID NO: 5 - DNA, 26 bp, Artificial Sequence
  PCR primer
cacagggtactttattgatggtacat

SEQ ID NO: 6 - DNA, 20 bp, Artificial Sequence
  PCR primer
agcaaggcattcctcttgaa

SEQ ID NO: 7 - DNA, 20 bp, Artificial Sequence
  PCR primer
ctacaggcagccaaatggtt

SEQ ID NO: 8 - DNA, 20 bp, Artificial Sequence
  PCR primer
tgcaccaccaactgcttagc

SEQ ID NO: 9 - DNA, 21 bp, Artificial Sequence
  PCR primer
ctcatgaccacagtccatgcc

SEQ ID NO: 10 - protein, 326 aa, Homo Sapiens
MSNGYEDHMAEDCRGDIGRTNLIVNYLPQNMTQDELRSLFSSIGEVESAKLIRDKVAGHSLGYGFVNYVTAKDAERAINTLNGLRLQSKTIKVSYARPSSEVIKDANLYISGLPRTMTQKDVEDMFSRFGRIINSRVLVDQTTGLSRGVAFIRFDKRSEAEEAITSFNGHKPPGSSEPITVKFAANPNQNKNVALLSQLYHSPARRFGGPVHHQAQRFRFSPMGVDHMSGLSGVNVPGNASSGWCIFIYNLGQDADEGILWQMFGPFGAVTNVKVIRDFNTNKCKGFGFVTMTNYEEAAMAIASLNGYRLGDKILQVSFKTNKSHK

SEQ ID NO: 11 - DNA, 6075 bp, Homo Sapiens
ggtcgtgcgcgctgaggaggagccgctgccgccgtcgccgtcgccgccaccgccgccaccgctaccgaggccgagcggagccgttagcgccgcgccgccgccgcctcccgcccgccccggagcagccccgggcccgcccgcccgcatccagatttttgaaaaatacaatgtctaatggttatgaagaccacatggccgaagactgcaggggtgacatcgggagaacgaatttgatcgtcaactacctccctcagaacatgacccaggatgagttacgaagcctgttcagcagcattggtgaagttgaatctgcaaaacttattcgggataaagtagcaggacacagcttgggctatggctttgtgaactacgtgaccgcgaaggatgcagagagagcgatcaacacgctgaacggcttgaggctccagtcaaaaaccattaaggtgtcgtatgctcgcccgagctcagaggtgatcaaagacgccaacttgtacatcagcgggctcccgcggaccatgacccagaaggacgtagaagacatgttctctcggtttgggcggatcatcaactcgcgggtcctcgtggatcagactacaggtttgtccagaggggttgcgtttatccggtttgacaaacggtcggaggcagaagaggcaattaccagtttcaatggtcataaacccccaggttcctctgagcccatcacagtgaagtttgcagccaaccccaaccagaacaaaaacgtggcactcctctcgcagctgtaccactcgccagcgcgacggttcggaggccccgttcaccaccaggcgcagagattcaggttctcccccatgggcgtcgatcacatgagcgggctctctggcgtcaacgtgccaggaaacgcctcctccggctggtgcattttcatctacaacctggggcaggatgccgacgaggggatcctctggcagatgtttgggccgtttggtgccgtcaccaatgtgaaagtgatccgcgacttcaacaccaacaagtgcaaagggtttggctttgtgaccatgacaaactatgaagaagccgcgatggccatagccagcctgaacggctaccgcctgggggacaaaatcttacaggtttccttcaaaaccaacaagtcccacaaataactcgctcatgcttttttttgtacggaatagataattaagagtgaaggagttgaaacttttcttgttagtgtacaactcattttgcgccaattttcacaagtgtttgtctttgtctgaatgagaagtgagaaggtttttatactctgggatgcaaccgacatgttcaaatgtttgaaatcccacaatgttagaccaatcttaagtttcgtaagttatttcctttaagatatatattaaacagaaatctaagtagaactgcattgactaaccagtccctctggatggtggtgaacctgaagcatgctttaacctctaagactgtctaacacgcgtttcattcaatgtctccacagactgggtagcaaaaaaatcaccttttagttttagtttttaatctaaagatgttagacagatgctgagtgtgcgttttctcaaccgcttcaacattgtaagcgatgtatgctttggttgacaggaagttccttttccaggcaggtcccgttgccacctcctgctcactcagtcccgggctctgccgagtggtcctgggaatggcggcgggcccgtccagcgtgggccaccactggggccgggggccacgggctgcatgctgggcgggccctccagagaaggacacaaacgtgtttcgtaagcccaggcaccaatgggaatggaccaaagagtttcagggaaactccagtatattccagagtcagatctaagctccaggcacgcctgaagatgtgttgctactctgacatcccgagtttctgtccacacattgcatgcacagcgccccacacattggatactgttgttcacgataatttctcccgttttccagagcatttaacatagcttggaggcgtaaaatggctctgtattttaataacacagaaacatttgagcattgtatttctcgcatcccttctcgtgagcgcttagacctttttctattttagtcggattttgttttggaattttgcttttgtatgaacactcagcagaaaagtacttacttcttgccagttatctattaaccaaaacctttgatttgtagttttaaagattaaccctcaaagttctcttcataactgccttgacattttgggttgttctgttctgttaattttcttttgcttttttgtgttttttgtttgtttttacttttgcatttaagaccattaaatttgattttgttttgctcgaattttgttttgttttttattttaccttttctttctttttggctagggaaggtgcaggtggcccagcattcagggaggagtcgtaagatcttaagaaaccaatccttgcctcaagcaaaagcatttctgaatctgtgacgcaagaatgtgcagttacaggctggtggcttttaaaccaggagcccggaggaagggtgaaagagaaagcctgtgaaataggcagggccagatcacccaaaactcctcaggactgggatctggcgtttataaataactagtttacagagagaatcacaaacaggataacttagtaccagcagtttttaaccttgacgtgagactaaaacgtgaccgtaggctgttttttagttattgctctcatgagatgatggctgtatttatctgtttatttatacctatttatgtatttatttattgaagtgtgaaattgagcaataggcaggcaccaccgtccccagagcaggtcagcgtctcgagaggcccctggacaattgaggatgcccatcccctcccctttccctgatcttttactgaggggctgtgtgcgcgatccttgcaaattgatgatgttgccatccgtacccaggctgtgtctcataaaagtcggcctggtgccagagaggacctcctttctcccacagaatcccagatcctcaggaaaagccaaaaccgaggcccattgcccggattcgacacaaaagagggtccctgctctgttgcccgagagcagtctgcatcctgggaccagaatgctttcctggaaaaagaagcctttcaggtttccctgggccagcatcttctgatggaaggtgggagccaacacccttctgatggaaggtgggagccaacacccttctgatggaaggtgggagccaacacccttctgatggaaggtgggagccaacacccttctgatggaaggtgggagccaacacccttctgatggaaggcgggattcccgcttctgaaactccccctggagtctcactcccacacatgcccatagctagcattcaacagagaactctgtcttaagcttcaactgtgaaaatgatgacgggcttgtagcacctcagcttctttcctcgcccccttttatctgaatcctatcaattattctgatgctgggacaggtgagaagaaactgtgaagtatatgagcctttgaaagttccctgaagtttctcagttcaggaacattctcattgtatgtggtctccgctgtttgaacagccttctagctaaaaaattccaaagcctttatttgggagtcttagcttgcaagcttgtggaaggatttagcttaacaactgtcactcctgaaaagcaatctctgttccatcaaggttctagttgctggccctgtgtcctcaaagttcattacatcttatcaaggcctgtttgcaaaggggagatcccttttcttaaaaaaggctcaacccaaaagaaaccatttcttaaaaaattttacatagatcagttgtatttctatttagcaaaaatgagtgctctgcttttatttgggaatttcgatgaaaaagcgttcagagtagataatgttcatttatcaaaaatctggtttgggaaataccaaagaggctttgattgaattcccttttgacccgtgtgtaacttcctctggtagttagaccccaggcagctccgaatttgtgaacctgcttcctgatgaattctcccttgttcccccttggctctgccattatttcgttttcagtgtaatttgccaagccgcagttttctgtgctggctgtgcctctagtcgcagctctgtgactgattccctcccgggtgctgagtcccctccccggccaccatcctgcgtgaatatcctgaaattcatgggcttcctcgggggcccgcccgcaggtggtgctgggtgggttccgccaccttctcctggaaggtgagccttttcctggccaagggcagctgccttaacctctgagagtctgcgcttggccttagtcctggagacccagcctccagggactgaaccgtgctgctgttgggagccaagaccggccctttggagccggcagcccaggggtccctgctggatcagagaaatagaagcacccgaagacggttagtggcaattccttgacccggtttgcttccaaatgaaggccatttgtccaccaggcattgaaaagacatgacttacccagtccggcatcggacttgaaaaatcgaaattgacatcactcagctgttacatttcacatccgattcagcccccttttatttccatgtgcttttcgcagccttcctgtgttggatgaaagagaataagaattcagctgacaggaggcctctatcctgtccctccaccccaccctccacctcaatcccctcccatcttccccagacctacctcacctactaggacctgaggcagctccttagcagagacccctggggtttagctgactctgggggtcaggggttcctgtccccaaacttcgcaagacagccctgaagtcacaagtgctttcttttaagtggcattggcaattggcgtgtaatgatggcagtagaatctgaatctcggatcccaggcagggttcacatttccaaacctttttgatttcccctgacctctaatggctggatcctatttttctacaacctttcagtgacatcgttcaggtttctttcttggccatttaaaaaaacaaattttttttttctcacttgtaagtcaccgccagtacctaagttaggctaacggagactttgacaggactggatttttcttccaccagaagagaagccttttccgttggtttggggccacctctttgaccatgaccatgtgatgttccgtttacagtgacttgctttgggggaggggaggctctcttaaccgattcccatgttgtacagtagatggttagaccttttgtatattagtgtgttttaagttattgatttgttttatataaaataatttatttttcaggtgccatttttcattttaactttgtttttacatgggtttgttttcaataaagtctgacactggtgtccaaaagtcaacaataaaatgaatcccattgtgttcttttgaagatgcctatgtaacttttaagctttttaaattattttcagaaaaaaaaaaagaaaagcccttatcagttttccatcagcccattgcctttttattttttttttttaatccttgtgaataaatgttctttagtgttttaggaggaaaaagcaaacctagattttgataacccagaagacttcagattaataaagaagctttgaaagaagaccatttttcaaaattttagtgaagtgtgaatattttttgtcaatggctttctcaaagagaatgaaacttttgcaccattttcagagtttttatagagatgccaaattgatatatttacatgtaatggaaacatgaaaaagttttattaaacaattgttcatagctgtgtagacattttaattcagtttccaaagctctcaaaaaatcgtatttttgaagtacggagtgatgcggtttggggcgtggcttacagttccaacgactcaattgtcccgatactcagttctttctacaggtatcaggttcgtgttaaacgctgtatgttaactatgactggaattctgtgatattttggtaataaatgaagtgggatcattgcgaaaaaaaaaaaaaaaaaa

SEQ ID NO: 12 - DNA, 20 bp, Artificial Sequence
  Synthetic sequence
ctccacccacacattcctgg

SEQ ID NO: 13 - DNA, 40 bp, Artificial Sequence
  Synthetic sequence
ctccacccacacattcctggctccacccacacattcctgg

SEQ ID NO: 14 - DNA, 60 bp, Artificial Sequence
  Synthetic sequence
ctccacccacacattcctggctccacccacacattcctggctccacccacacattcctgg